(19) United States
(12) Patent Application Publication
(10) Pub. No.: US 2022/0096562 A1
(43) Pub. Date: Mar. 31, 2022

(54) OSTEOBLAST CELL-MIXTURE, AND IMPLEMENTATIONS THEREOF

(71) Applicant: REGROW BIOSCIENCES PRIVATE LIMITED, Mumbai (IN)

(72) Inventors: Satyen SANGHAVI, Mumbai (IN); Vinayak KEDAGE, Pune (IN)

(21) Appl. No.: 17/428,592

(22) PCT Filed: Feb. 7, 2020
(86) PCT No.: PCT/IN2020/050122

§ 371 Date: Aug. 4, 2021

(30) Foreign Application Priority Data
Feb. 8, 2019 (IN) 201921005149

(51) Int. Cl. A61K 35/32; A61K 9/00

(52) U.S. Cl. CPC A61K 35/32; A61K 9/0019

(57) ABSTRACT

The present disclosure provides a method for obtaining osteoblast cell-mixture which can be used for transplantation of osteoblast cells in a subject. The present disclosure further discloses a method for delivering osteoblast cells into a subject. The method for obtaining osteoblast cell-mixture as disclosed herein is devoid of any additives like calcium chloride and aprotinin. The method for delivering osteoblast cells as disclosed herein provides bone regeneration in the subject.

### FIELD OF INVENTION

The present disclosure broadly relates to the field of therapeutic composition, and in particular discloses a composition comprising osteoblast cells and method of administering the same.

### BACKGROUND OF INVENTION

Bones are an integral part of the musculoskeletal system. While it is the largest connective tissue, its main functions include support, strength, movement and protection. Bone formation and maintenance is a very specialized process, continuously happening in any healthy individual. Bone tissue is made up of at least three types of cells and extracellular matrix (ECM). The maturity and hardness of bone are dependent on composition of the cells and ECM.

The ongoing process of maintenance of bone is called remodelling, during which, the new bone-forming progenitor cells i.e., osteoblasts are recruited, which eventually mature into osteocytes. Osteocytes have a specified lifespan, after which they are resorbed by another special type of cells called osteoclasts. During homeostasis, healthy bone tissue is continuously formed and resorbed with normal structure and function.

The ECM is composed of collagen and minerals like calcium and phosphate. Tensile strength, and compressive strength of a bone is dependent on the type of collagen, and the minerals' proportion.

Bones are supposed to be strong and supportive as well as protective. Any condition that would lead to bones becoming weak, fragile, or brittle, would be considered as or would result in diseased conditions of the bones. Mineral imbalance, metabolic and hormonal disturbance, cellular insufficiency and/or simple trauma are known reasons for diseased condition of bones. While some conditions are genetic, many are due to medications, nutritional deficits etc Inflammatory disorders as well as malignancies involving bones are also known. Overall, there could be structural deformities (e.g., short limbs or scoliosis); nutritional deficits (e.g., rickets, Paget's disease); compromised bone density (e.g., osteoporosis); brittle bones (e.g., osteopetrosis); infection (e.g., osteomyelitis); inflammatory diseases of bones and joints (e.g., rheumatoid arthritis); degenerative conditions (e.g., osteoarthritis); vascular deficits (e.g., osteonecrosis of hip) and drug-induced abnormalities (e.g., osteonecrosis of jaw).

The composition of minerals, collagen (overall ECM), and homeostasis of remodelling are the determinants of health of a bone. Thus, any derailed metabolic process that would result in abnormal mineral composition of bone would be responsible for diseased condition. In the same manner, derailed bone remodelling, as a result of, for example, inadequate recruitment of osteoblasts and/or increased or uncontrolled activity of osteoclasts, would result in a diseased condition. Traumatic insults like fractures; especially when they do not heal in stipulated time frame can be considered diseased conditions. Cysts and benign tumors, and fibrous dysplasia like conditions that occupy and damage bone from within are another type of bone disease conditions. Age-related degenerative changes that progressively damage the collagen, and hence cartilage lead to osteoarthritis; while rheumatoid arthritis is a typical autoimmune disease. Few more diseases that require urgent attention of the researchers are: (i) Avascular necrosis (AVN); (ii) Non-union fractures; (iii) bone cysts/fibrous dysplasia; and (iv) Bone loss and osteonecrosis in oral and maxillofacial (OMF) conditions.

The concept of regenerative medicine encompasses various stem cells derived from various sources, biological scaffolds (membranes etc.), Platelet-Rich Plasma, Platelet-Rich Fraction and the like. While most of them can be available at the bedside, collectively called as Bone-Marrow-Aspirate-Concentrate (BMAC), their clinical use is not approved for any of the conditions. BMAC basically is a source of heterogenous population of progenitor cells and its composition (types of cells, their cell counts etc.) but it cannot be ascertained before each application. In absence of this, most of the positive effects are restricted to the paracrine activity, that which is has the limitation of being time-bound. Thus, repair of the lost bone, resumption of bone-remodelling, structural integrity and biomechanics of the affected joint or part of body cannot be achieved.

U.S. Pat. No. 5,824,084A discloses a method providing a bone marrow aspirate suspension and passing the bone marrow aspirate suspension through a porous, biocompatible, implantable substrate to provide a composite bone graft having an enriched population of connective tissue progenitor cells.

US20180195043 discloses a method for generating an osteoblast that is applicable to repair of a bone defect due to various tumors, injuries, surgeries, etc., and due to treatment for bone resorption typified by a periodontal disease, bone fracture, osteoporosis, etc., and that has a low risk of carcinogenesis.

All the above-mentioned conditions are characterized by deficiency or imbalance of vasculature and blood supply, accumulation of unwanted debris and lack of recruitment of committed and differentiated progenitor cells. Obviously, it is logical that the treatment modality should revolve around and aim at correcting these faults and bring about repair and restoration of function. Therefore, there still persists a problem in the field to which effective solution in terms of an effective composition comprising osteoblast and an associated transplantation technique is lacking.

All publications have been added fully to the disclosure by reference.

### SUMMARY OF THE INVENTION

In an aspect of the present disclosure, there is provided a method for preparing an osteoblast cell-mixture, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; and (f) mixing the second mixture of step (c) with the third mixture of step (e), to obtain an osteoblast cell-mixture.

In another aspect of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; and (g) delivering the osteoblast cell-mixture at a site in a subject.

In yet another aspect of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) adjusting position of the subject into a gravity-dependent position; (j) injecting the osteoblast cell-mixture of step (f) into the defect area and allowing the osteoblast cell-mixture to form a gel network; and (k) applying standard medical procedures for suturing, to deliver osteoblast cell-mixture into the subject.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF ACCOMPANYING DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts clotted bone marrow biopsy, in accordance with an embodiment of the present disclosure.

FIG. 2 depicts pipetting of bone marrow biopsy, in accordance with an embodiment of the present disclosure.

FIG. 3 depicts a sample filtered with 100 μM cell strainer, in accordance with an embodiment of the present disclosure.

FIG. 4 depicts bone marrow derived stem cells (BM-MSCs) before staining, in accordance with an embodiment of the present disclosure.

FIG. 5 depicts Crystal violet staining showing stained BM-MSCs colonies in Petri plates, in accordance with an embodiment of the present disclosure.

FIGS. 6A and 6B depict bone marrow-derived mesenchymal stem cells (BM-MSCs) derived colony forming units-F (CFU-F) colonies from clotted bone marrow (BM) sample at 100× magnification, in accordance with an embodiment of the present disclosure.

FIG. 7 depicts immunophenotypic results using Flow cytometry for BM-MSCs at 14±3 days of culture showing CD90 and CD105 positive expression and CD34 negative expression (Representation of Sample No. 05), in accordance with an embodiment of the present disclosure. P1 depicts forward scattering v/s side scattering data, P2 depicts results for CD90, P3 depicts results for CD105, and P4 depicts results for CD34.

FIG. 8 depicts immunophenotypic results using Flow cytometry for BM-MSCs at 14±3 days of culture showing CD73 positive expression and HLA-DR negative expression (representation of Sample No. 05), in accordance with an embodiment of the present disclosure. P1 depicts forward scattering v/s side scattering data, P2 depicts results for FITC, P3 depicts results for CD73, and P4 depicts results for HLA-DR.

FIG. 9 depicts a flowchart for the process of isolating fibrinogen, in accordance with an embodiment of the present disclosure.

FIG. 10 depicts cord blood and maternal blood plasma as starting material for preparation of fibrinogen and thrombin, in accordance with an embodiment of the present disclosure.

FIG. 11 depicts saturated ammonium sulphate prepared in sterile distilled water, in accordance with an embodiment of the present disclosure.

FIG. 12 depicts addition of protein precipitation solution to mixed cord blood plasma and maternal blood plasma, in accordance with an embodiment of the present disclosure.

FIG. 13 depicts precipitation of proteins for 5-20 minutes, in accordance with an embodiment of the present disclosure.

FIG. 14 depicts centrifugation of precipitated proteins at 3000-5000 rpm for 5-10 minutes, in accordance with an embodiment of the present disclosure.

FIG. 15 depicts the discarded supernatant, in accordance with an embodiment of the present disclosure.

FIG. 16 depicts the protein pellet, in accordance with an embodiment of the present disclosure.

FIG. 17 depicts protein pellet dissolved in Dulbecco's phosphate-buffered saline (DPBS)/water for injection (WFI)/saline, in accordance with an embodiment of the present disclosure.

FIG. 18 depicts centrifugation at 1200-2500 rpm for 5-10 minutes, in accordance with an embodiment of the present disclosure.

FIG. 19 depicts supernatant of dissolved proteins (containing fibrinogen) collected in separate tube, in accordance with an embodiment of the present disclosure.

FIG. 20 depicts thrombin isolated using a process, in accordance with an embodiment of the present disclosure.

FIG. 21 depicts the process flow of preparing plasma rich platelet (PRP), in accordance with an embodiment of the present disclosure.

FIG. 22 depicts the process flow of preparing the osteoblast cell-mixture using PRP obtained in accordance with an embodiment of the present disclosure.

FIG. 23 depicts the process flow of preparing platelet lysate (PL), in accordance with an embodiment of the present disclosure.

FIG. 24 depicts the process flow of preparing the osteoblast cell-mixture using the PL, in accordance with an embodiment of the present disclosure.

FIG. 25 depicts a picture of sedimentation of umbilical cord blood to obtain platelet rich plasma (PRP) from umbilical cord blood, in accordance with an embodiment of the present disclosure.

FIG. 26 depicts a picture of sedimentation of maternal blood to obtain platelet rich plasma (PRP) from maternal blood, in accordance with an embodiment of the present disclosure.

FIG. 27 depicts a picture of centrifuged PRP after mixing of Platelet Rich Umbilical Cord Blood Plasma and Platelet Rich Maternal Blood for preparation of PRP, in accordance with an embodiment of the present disclosure.

FIG. 28 depicts a picture of 10% calcium gluconate (at least one activator), in accordance with an embodiment of the present disclosure.

FIG. 29 depicts a picture of live cultured Human Cell Suspension, in accordance with an embodiment of the present disclosure.

FIG. 30 depicts a picture of prepared PRP, in accordance with an embodiment of the present disclosure.

FIG. 31 depicts a mixing of Calcium Gluconate (D)+Live cultured Human Cell Suspension (E)+Prepared PRP (F) in Mixing vial, in accordance with an embodiment of the present disclosure.

FIG. 32 depicts aspiration of mixture from Mixing Vial with the help of Syringe, in accordance with an embodiment of the present disclosure.

FIG. 33 depicts the formation of gel, in accordance with an embodiment of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

For the purposes of the present document, the term “umbilical cord blood” (UCB) as used herein means the blood that remains in the placenta and in the attached umbilical cord after childbirth. Cord blood is collected because it contains stem cells, which can be used to treat hematopoietic and genetic disorders. Generally, a lot of this rich biological resource is discarded. The definition is not meant to be restricted to one species of subject, it generally covers vertebrates. The subject which has been considered in the present disclosure is human. Specifically, umbilical cord blood is collected from umbilical vein of a newly born baby/infant.

The term “maternal blood” as used herein means the blood collected from a mother pre- and post-delivery. The maternal blood (MB) collection may take place at a time immediately before/after cord blood collection, at the time of admission for delivery (after initiation of labour) or before transfusion/infusion of any intravenous fluid (colloids/crystalloids/blood products). The definition is not meant to be restricted to one species of subject, it generally covers vertebrates. The subject which has been considered in the present disclosure is human. Specifically, maternal blood refers to blood from mother after delivery of baby, but within 7 days from the delivery.

The term “platelet lysate” (PL) as used herein means cell lysates produced from regular platelet transfusion units by lysis.

The term “platelet” as used herein refers to cells which are small a-nucleated structures of hematopoietic origin which contribute to homeostasis and wound healing by secreting growth factors and cytokines. They are produced by the fragmentation of megakaryocytes and released into the bloodstream, where they circulate for 7-10 days before being replaced.

The term “umbilical cord blood” (UCB) as used herein means the blood that remains in the placenta and in the attached umbilical cord after childbirth. Cord blood is collected because it contains stem cells, which can be used to treat hematopoietic and genetic disorders. Generally, a lot of this rich biological resource is discarded. The definition is not meant to be restricted to one species of subject, it generally covers vertebrates. The subject which has been considered in the present disclosure is human.

The term “bone regeneration” refers to the generation of bone tissue that is required to correct the defective condition. The time may vary depending upon the extent of damage to the bone. Bone regeneration may start around 5 weeks but will depend on case to case basis, for instance it and may vary from 5-6 weeks to 3-4 months.

The term “dual-syringe device” or “dual-syringe applicator” refers to any device which allows application of ingredients from physically separable vials into a site. The site in this context refers to an area in a subject that is used for surgical intervention. “Affected area” refers to the area which is affected by a disorder, “Defect area” refers to an area which has the defect and is used as a target in case of surgical intervention. Affected area is used to approach the defect area in a subject. The term “curettage” refers to a medical procedure in which tissue is scrapped or scooped using a curette. The term “necrosed bone” refers to bone tissue which has been affected by necrosis, one of the causes being interruption of blood supply.

The term “osteoblast cell suspension” is contemplated to comprise osteoblast cells. The osteoblast cell suspension is used to prepare “osteoblast cell-mixture”.

The term “autologous osteoblast cells” refers to osteoblast cells that has been obtained by differentiating autologous mesenchymal cells.

The term “gravity dependent” refers to a position in which force of gravity can be used to facilitate a step required during any kind of treatment.

The term “standard medical procedures” refers to well-known procedures that are followed during the treatment process.

The term “C-Arm” refers to a medical imaging device that is based on X-ray technology and can be used flexibly. The term “C-Arm guidance” refers to a step which has been performed under the guidance of C-Arm device.

The term “necrotic area” refers to an area affected by necrosis. The term “heat shock proteins” or “HSP” is intended to cover all the heat shock proteins that is known in the art ranging from 10-100 kDa. HSP 70 is one such heat shock protein with 70 kDa molecular weight.

Abbreviations as used herein include, α-MEM—α Minimum Essential Medium, DMEM—Dulbecco's Modified Eagle Medium, IMDM—Iscove's Modified Dulbecco's Medium, EMEM—Eagle's Minimum Essential Medium, FGF—Fibroblast Growth Factor, TGF—Transforming Growth Factor, IGF—Insulin-like growth factor 1, VEGF—Vascular endothelial growth factor, PDGF—Platelet-derived growth factor, BMP-2—Bone Morphogenic Protein-2, CD90—Cluster of Differentiation 90, CD73—Cluster of Differentiation 73, CD105—Cluster of Differentiation 105, CD34—Cluster of Differentiation 34, HLA-DR—Human Leukocyte Antigen—DR isotype, Umbilical Cord Blood (UCB), Maternal Blood (MB), Platelet-rich Plasma (PRP), platelet lysate (PL), Mesenchymal Stem Cells (MSCs), OCT-4—octamer-binding transcription factor 4, Sox-2—Sex determining region Y box 2, ALP—Alkaline phosphatase, bone alkaline phosphatase

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

The goal of treatment in the conditions mentioned in the background section, especially, AVN, should be to achieve the following steps:

1) Revascularization—The step of core decompression is known to re-initiate required blood-flow through new blood vessel networks. Core decompression is performed to improve the repair in the ischemic osteonecrotic segment particularly at earlier stages of necrosis before mechanical failure of the femoral head has occurred. New vessels and bone cells arrive in the dead bone along the decompression tunnel. In an adult, hematopoietic red marrow is normally absent in the femoral head, but red marrow containing stem cells persist in the proximal shaft of the femur. These cells elicit formation of new blood vessels by the presence of endothelial cell progenitors or hemangioblasts in this cell fraction. Endothelial progenitors actively engage in vasculogenesis in tissue devoid of vessels, and in neoangiogenesis from the pre-existing capillaries. Besides the generation of new capillaries, the growing endothelia enhance mobilization and growth of mesenchymal progenitors through angiopoietin 1-Tie2 pathway, which generate pericytes and vascular mural cells required for new vessel growth and stabilization. However, this fraction of cells is insufficient to ensure complete repair in most cases. Hence, treatment modalities should be to stimulate and guide bone remodeling to creeping substitution to preserve the integrity of the femoral head. The present disclosure provides a solution to preserve the integrity of femoral head.

2) Curettage—This is the most essential step during which, the present disclosure targets complete debridement of the necrotized bone. This ensures two things. One, least chance of blood clot formation and hence, of compromised new bone quality. Second, enough space for the cell-gel mixture implant.

The second step of curettage is performed to debride the necrotic tissue to create space for the replacement of dead bone tissue with bone marrow mesenchymal stem cell-derived osteoblasts. The process involves complete removal of the necrotic tissue, where the diseased area is thoroughly curetted by debriding the head and sclerotic bone. The process of curettage of the necrotic bone is necessary to counter the faulty process of cell repair that is marked by new bone formation over the dead bone trabeculae.

3) Implant of committed, differentiated osteoblasts: it ensures effective formation of new bone that would be like native bone but devoid of additional cells which serve as are contaminating with respect to the purpose of invention.

The third step involves delivery of in vitro bone marrow mesenchymal stem cell-derived osteoblasts in a fibrin gel system. For the repair and replacement of injured tissue with biological substitutes, the following factors need to be considered. Firstly, appropriate cells must be present to give rise to structural tissue. Secondly, appropriate growth factors and stimuli must exist for the cells to proceed down the proper lineage. Finally, a scaffolding matrix must act as a building block for cellular attachment, differentiation and maturation into the desired tissue.

This unmet medical need gave way to the present disclosure which discloses a method to prepare adult, autologous, live, transplantation-ready cultured osteoblast cells mixed with appropriate gel mixture. The salient features of the present disclosure are: (i) Autologous cells—ensures safety with near-minimum chances of GvHD (Graft vs host disease); and (ii) Live-cultured cells—assures cell count, with characterization. Thus, no ambiguity in cell type and cell count since, osteoblast cells can differentiate only into osteocytes.

The present disclosure makes use of fibrin and thrombin system to arrive at a composition which can be delivered to a subject.

Osteoblasts also produce alkaline phosphatase which is an enzyme that is involved in the mineralization of bone. It is an early marker of osteoblast differentiation and its increased expression is associated with the progressive differentiation of osteoblasts. Injecting cultured osteoblasts have only two specific transcripts, one encoding Cbfa1 and other encoding osteocalcin, an inhibitor of osteoclast function that is only expressed when these cells are completely differentiated. Osteoblasts injected are already immature bone tissue that leads to mature bone formation at the decompression site. The injection of the cultured osteoblasts prevents the loss of structural integrity of the subchondral trabeculae thus aids in restoration of the bone mass.

In earlier attempts, with different types of cells, loss of cell suspension during or soon after implant has been a major limitation. Hence, the present disclosure discloses a unique cell-delivery system that makes use of physiological property of thrombin-fibrin network. When the patient-specific cell suspension gets mixed with this special delivery system, soon after implantation (within 3-6 minutes), it would transform into a well-set gel, occupying the space where it is implanted. The implanted osteoblast cells are enough to execute the expected repair through integration with native bone, reestablishment of bone remodeling, resuming joint biomechanics and activity, movement and strength that is continuous and ongoing.

One of the biggest cell therapy areas in tissue engineering is defined by either (1) in vivo, that is, by stimulating the body's own regeneration response with the appropriate biomaterial, or by (2) ex vivo, that is, cells can be expanded in culture, attached to a scaffold and then re-implanted into the host. Depending on the source, cells may be heterologous (different species), allogeneic (same species, different individual) or autologous (same individual). Autologous cells are preferred as they will not evoke an immunologic response and thus, the deleterious side effects of immunosuppressive agents can be avoided. In addition, the potential risks of pathogen transfer are also eliminated. When engineering bone tissue substitutes, mechanical stability, osteo-conductivity, osteo-inductivity, osteo-genicity and ease of handling must be well balanced in order to properly meet any clinical needs.

Typically, bone tissue engineering requires not only living cells but also the use of scaffolds, which serve as a three-dimensional environment for the cells. Scaffolds for engineering bone should be: (i) biocompatible (non-immunogenic and non-toxic); (ii) absorbable (with rates of resorption commensurate with those of bone formation); (iii) preferably radiolucent (to allow the new bone to be distinguished radiographically from the implant); (iv) osteoconductive; (v) easy to manufacture and sterilize; and (vi) easy to handle in the operating theater, preferably without preparatory procedures (in order to limit the risk of infection). Three-dimensional scaffolds for bone tissue regeneration require an internal microarchitecture, specifically highly porous interconnected structures and a large surface-to-volume ratio, to promote cell in-growth and cell distribution throughout the matrix. Pore sizes, Particle size, shape and surface roughness affect cellular adhesion, proliferation and phenotype. Specifically, cells are sensitive and responsive to the chemistry, topography and surface energy of the material substrates with which they interact. In this respect, the type, amount and conformation of specific proteins that adsorb onto material surfaces, subsequently modulate cell functions.

Therefore, the present disclosure has been made in view of the above aspect, and it is an object of the present disclosure to provide a semi-solid osteoblast composition containing fibrin glue comprising fibrinogen and thrombin for bone formation and a method for preparing the same. The present disclosure aims for having no clinical graft rejection via injection of an osteoblasts with fibrinogen and thrombin mixture into a defect site where bone formation is sought, and capable of achieving effective and rapid bone formation via injection of a composition which was shaped to a certain extent, in order to ease problems associated with bone tissue formation.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

In an embodiment of the present disclosure, there is provided a method for preparing an osteoblast cell-mixture, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; and (f) mixing the second mixture of step (c) with the third mixture of step (e), to obtain an osteoblast cell-mixture. In another embodiment, the osteoblast cell suspension is prepared from a mesenchymal stem cell suspension; and said mesenchymal stem cell suspension is obtained from a clotted bone marrow sample. In yet another embodiment, the first mixture comprises 100-600 IU/ml thrombin. In an alternate embodiment, the third mixture comprises 20-100 mg/ml of fibrinogen.

In an embodiment of the present disclosure, there is provided a method for preparing an osteoblast cell-mixture, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; and (f) mixing the second mixture of step (c) with the third mixture of step (e), to obtain an osteoblast cell-mixture, wherein the osteoblast cell suspension is prepared from a mesenchymal stem cell suspension, and said mesenchymal stem cell suspension is obtained from a clotted bone marrow sample, and wherein the platelet lysate comprises a lysate obtained from a mixture of an umbilical cord blood (UCB) derived platelet rich plasma and a maternal blood (MB) derived platelet rich plasma, and the mixture of an umbilical cord blood (UCB) derived platelet rich plasma and a maternal blood (MB) derived platelet rich plasma comprises 0.3×10⁹ to 1.5×10⁹ platelets/ml.

In an embodiment of the present disclosure, there is provided a method for preparing an osteoblast cell-mixture as described herein, wherein the first nutrient medium of step (b) or the second nutrient medium of step (e) comprises at least one medium selected from a group consisting of DMEM, αMEM, IMDM, and combinations thereof.

In an embodiment of the present disclosure, there is provided a method for preparing an osteoblast cell-mixture as described herein, wherein the osteoblast cell-mixture is used in transplantation of osteoblast cell suspension into a subject.

In an embodiment of the present disclosure, there is provided a method for preparing an osteoblast cell-mixture, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; and (f) mixing the second mixture of step (c) with the third mixture of step (e), to obtain an osteoblast cell-mixture, wherein the osteoblast cell suspension comprises osteoblast cells in a range of 12×10⁶ cells to 60×10⁶ cells.

In an embodiment of the present disclosure, there is provided a method for preparing an osteoblast cell-mixture as described herein, wherein the second mixture and the third mixture is mixed in a ratio in a range of 1:0.5 to 1:2. In another embodiment of the present disclosure there is provided a method for preparing an osteoblast cell-mixture as described herein, the second mixture and the third mixture is mixed in a ratio of 1:1.

In an embodiment of the present disclosure, there is provided a method for preparing an osteoblast cell-mixture as described herein, wherein fibrinogen is obtained from a mixture of umbilical cord blood (UCB) plasma and maternal blood (MB) plasma. In another embodiment there is provided a method for preparing an osteoblast cell-mixture as described herein, thrombin is obtained from a mixture of umbilical cord blood (UCB) plasma and maternal blood (MB) plasma.

In an embodiment of the present disclosure, there is provided a method for preparing an osteoblast cell-mixture, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; and (f) mixing the second mixture of step (c) with the third mixture of step (e), to obtain an osteoblast cell-mixture, wherein the osteoblast cell-mixture forms a gel network in a time in a range of 2 seconds-6 minutes. In another embodiment of the present disclosure, the osteoblast cell-mixture forms a gel network in a time in a range of 2 minutes-5 minutes.

In an embodiment of the present disclosure, there is provided a method for preparing an osteoblast cell-mixture, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; and (f) mixing the second mixture of step (c) with the third mixture of step (e), to obtain an osteoblast cell-mixture, wherein the osteoblast cell-mixture comprises autologous osteoblast cells.

In an embodiment of the present disclosure, there is provided a method for preparing an osteoblast cell-mixture, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; and (f) mixing the second mixture of step (c) with the third mixture of step (e), to obtain an osteoblast cell-mixture, wherein the method is used to correct conditions selected from a group consisting of non-union fracture, fibrous dysplasia, avascular necrosis, oral and maxillofacial fractures, and sinus lift.

In an embodiment of the present disclosure, there is provided a method for preparing an osteoblast cell-mixture, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; and (f) mixing the second mixture of step (c) with the third mixture of step (e), to obtain an osteoblast cell-mixture, wherein the first mixture comprises 100-600 IU/ml thrombin, and the third mixture comprises 20-100 mg/ml of fibrinogen, and the second mixture and the third mixture are mixed in a ratio of 1:1, and thrombin and fibrinogen are independently obtained from a mixture of umbilical cord blood (UCB) plasma and maternal blood (MB) plasma, and the osteoblast cell-mixture forms a gel network in a time in a range of 3-6 minutes, and the mixing of the second mixture and the third mixture is done by a dual syringe device.

In an embodiment of the present disclosure, there is provided a method for preparing an osteoblast cell-mixture, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; and (f) mixing the second mixture of step (c) with the third mixture of step (e), to obtain an osteoblast cell-mixture, wherein thrombin is obtained by a method comprising: (i) mixing umbilical cord blood plasma with maternal blood plasma to obtain mixture A; (ii) contacting saturated ammonium sulphate/ethanol to the mixture A in a ratio selected from a group consisting of 1:1, 2:1 and 3:1, wherein the ratio is between cord blood in mixture A and the saturated ammonium sulphate/ethanol, and allowed to precipitate plasma proteins for 5-20 minutes to obtain a mixture B; (iii) centrifuging the mixture B at 3000-5000 rpm for 5-10 minutes to separate protein precipitate in form of a pellet from a supernatant; (iv) discarding the supernatant and adding 4-5 ml of a solution A to the pellet to obtain a pellet solution; (v) centrifuging the pellet solution, and collecting supernatant; (vi) lyophilizing the supernatant under vacuum conditions to obtain a lyophilized protein powder; (vii) dissolving the lyophilized protein powder in a solution B to obtain a protein solution; (viii) extracting prothrombin by ion exchange chromatography using diethyl aminoethyl (DEAE-IEC) followed by heparin affinity chromatography (A second DEAE-IEC step), followed by immobilized metal affinity chromatography (IMAC), and collecting prothrombin; (ix) activating prothrombin obtain crude thrombin and purifying crude thrombin by hydrophobic interaction chromatography (HIC), to obtain purified thrombin; (x) concentrating the purified thrombin, to obtain thrombin, wherein the solution A and solution B is independently selected from a group consisting of water, saline, and Dulbecco's phosphate buffer saline. In another embodiment, there is provided a method for preparing an osteoblast cell-mixture as described herein wherein fibrinogen is obtained by a method comprising: (i) mixing umbilical cord blood plasma with maternal blood plasma to obtain mixture A; (ii) contacting saturated ammonium sulphate/ethanol to the mixture A in a ratio selected from a group consisting of 1:1, 2:1 and 3:1, wherein the ratio is between cord blood in mixture A and the saturated ammonium sulphate/ethanol, and allowed to precipitate plasma proteins for 5-20 minutes to obtain a mixture B; (iii) centrifuging the mixture B at 3000-5000 rpm for 5-10 minutes to separate a pellet from a supernatant; (iv) lyophilizing the supernatant under vacuum conditions resulting in a lyophilized protein powder, wherein the lyophilized protein powder comprises fibrinogen wherein the solution A is selected from a group consisting of water, saline, and Dulbecco's phosphate buffer saline.

In an embodiment of the present disclosure, there is provided a method for preparing an osteoblast cell-mixture, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; and (f) mixing the second mixture of step (c) with the third mixture of step (e), to obtain an osteoblast cell-mixture, wherein the osteoblast cell suspension is obtained using a process comprising: (i) seeding in a culture flask, a mesenchymal stem cell suspension in a nutrient medium comprising 10% to 20% of a platelet lysate in the nutrient medium, to obtain culture flask-adhered mesenchymal stem cells; (ii) culturing the adhered mesenchymal stem cells in a nutrient medium comprising 10% to 20% of a platelet lysate in the nutrient medium; (iii) supplementing the nutrient medium of step (ii) with differentiation factors and growth factors to obtain a differentiation medium; (iv) complementing the differentiation nutrient medium of step (iii) with fresh differentiation nutrient medium comprising 10% to 20% of a platelet lysate, differentiation factors and growth factors to obtain a population of pre-osteoblast cells; (v) sub-culturing the population of pre-osteoblast cells of step (iv) for a time in a range of 12 to 15 days to obtain a P1 pre-osteoblast cells; (vi) expanding the P1 pre-osteoblast cells in a P1 expansion nutrient medium comprising 10% to 20% of a platelet lysate in the P1 expansion nutrient medium for a time in a range of 12 to 15 days to obtain expanded P1 pre-osteoblast cells, and sub-culturing the expanded P1 pre-osteoblast cells, to obtain a P2 osteoblast cells; and (vii) expanding the P2 osteoblast cells in a P2 expansion nutrient medium comprising 10% to 20% of a platelet lysate in the P2 expansion nutrient medium to obtain osteoblast cell suspension, and wherein the mesenchymal stem cell suspension is prepared using a process comprising: (1) obtaining a bone marrow sample, wherein the bone marrow sample comprises clotted bone marrow; (2) chopping the clotted bone marrow into pieces of at least 2 mm³ to obtain chopped clotted bone marrow; (3) contacting the chopped clotted bone marrow to at least one or combinations of enzymes in presence of a buffer to obtain a clotted bone marrow reaction solution; (4) incubating the clotted bone marrow reaction solution at a temperature in a range of 35° C. to 39° C. for a time of at least 20 minutes at a speed in a range of 100 rpm to 200 rpm to obtain incubated clotted bone marrow reaction solution; (5) contacting the incubated clotted bone marrow reaction solution of step (4) to a growth medium to obtain a suspension, wherein the growth medium is selected from a group consisting of α-MEM, DMEM, IMDM, F12, EMEM and combinations thereof; (6) mixing the suspension for a plurality of repeats; (7) filtering the suspension of step (6) with a cell strainer with a pore size in a range of 50 μm-100 μm to obtain a filtrate; (8) centrifuging the filtrate at 1300 rpm to 1800 rpm for a time in a range of 10 minutes to 15 minutes to obtain a cell pellet; and (9) dissolving the cell pellet with a nutrient medium to obtain a mesenchymal stem cell suspension, wherein the nutrient medium comprises 10% to 20% of a platelet lysate, and a medium selected from a group consisting of α-MEM, DMEM, IMDM, F12, EMEM and combinations thereof, and a plurality of factors selected from a group consisting of L-Glutamine, FGF, TGF, and wherein the at least one enzyme is selected from a group consisting of urokinase, collagenase, hyaluronidase, and combinations thereof, and the at least one protein is selected from a group consisting of heat shock proteins, and combinations thereof, and wherein the heat shock protein is heat shock protein 70 (HSP 70).

In an embodiment of the present disclosure, there is provided a method for preparing mesenchymal stem cells suspension from a clotted bone marrow, said method comprising: (1) obtaining a bone marrow sample, wherein the bone marrow sample comprises clotted bone marrow; (2) chopping the clotted bone marrow into pieces of at least 2 mm³ to obtain chopped clotted bone marrow; (3) contacting the chopped clotted bone marrow to at least one enzyme or at least one protein or combinations thereof in presence of a buffer to obtain a clotted bone marrow reaction solution; (4) incubating the clotted bone marrow reaction solution at a temperature in a range of 35° C. to 39° C. for a time of at least 20 minutes at a speed in a range of 100 rpm to 200 rpm to obtain incubated clotted bone marrow reaction solution; (5) contacting the incubated clotted bone marrow reaction solution of step (4) to a growth medium to obtain a suspension, wherein the growth medium is selected from a group consisting of α-MEM, DMEM, IMDM, F12, EMEM and combinations thereof; (6) mixing the suspension for a plurality of repeats; (7) filtering the suspension of step (6) with a cell strainer with a pore size in a range of 50 μm-100 μm to obtain a filtrate; (8) centrifuging the filtrate at 1300 rpm to 1800 rpm for a time in a range of 10 minutes to 15 minutes to obtain a cell pellet; and (9) dissolving the cell pellet with a nutrient medium to obtain a mesenchymal stem cell suspension, wherein the nutrient medium comprises 10% to 20% of a platelet lysate, and a medium selected from a group consisting of α-MEM, DMEM, IMDM, F12, EMEM and combinations thereof, and a plurality of factors selected from a group consisting of L-Glutamine, FGF, TGF, and wherein the at least one enzyme is selected from a group consisting of urokinase, collagenase, hyaluronidase, and combinations thereof, and the at least one protein is selected from a group consisting of heat shock proteins, and combinations thereof, and wherein urokinase is present in a range of 10,000 units to 30,000 units, collagenase type I-II is present in a range of 200 units to 500 units, and hyaluronidase type I-IV is present in a range of 200 units to 1000 units, and wherein the protein is heat shock protein 70 (HSP 70).

In an embodiment of the present disclosure, there is provided a method for preparing mesenchymal stem cells suspension from a clotted bone marrow, said method comprising: (1) obtaining a bone marrow sample, wherein the bone marrow sample comprises clotted bone marrow; (2) chopping the clotted bone marrow into pieces of at least 2 mm³ to obtain chopped clotted bone marrow; (3) contacting the chopped clotted bone marrow to at least one or combinations of enzymes in presence of a buffer to obtain a clotted bone marrow reaction solution; (4) incubating the clotted bone marrow reaction solution at a temperature in a range of 35° C. to 39° C. for a time of at least 20 minutes at a speed in a range of 100 rpm to 200 rpm to obtain incubated clotted bone marrow reaction solution; (5) contacting the incubated clotted bone marrow reaction solution of step (4) to a growth medium to obtain a suspension, wherein the growth medium is selected from a group consisting of α-MEM, DMEM, IMDM, F12, EMEM and combinations thereof; (6) mixing the suspension for a plurality of repeats; (7) filtering the suspension of step (6) with a cell strainer with a pore size in a range of 50 μm-100 μm to obtain a filtrate; (8) centrifuging the filtrate at 1300 rpm to 1800 rpm for a time in a range of 10 minutes to 15 minutes to obtain a cell pellet; and (9) dissolving the cell pellet with a nutrient medium to obtain a mesenchymal stem cell suspension, wherein the nutrient medium comprises 10% to 20% of a platelet lysate, and a medium selected from a group consisting of α-MEM, DMEM, IMDM, F12, EMEM and combinations thereof, and a plurality of factors selected from a group consisting of L-Glutamine, FGF, TGF, and wherein the mesenchymal stem cells are culture flask-adherent, nucleated, and stain with crystal violet stain, and the mesenchymal stem cells test positive in flow cytometry cell surface marker analysis for CD90, CD73 and CD105, and negative for CD34 and HLA-DR.

In an embodiment of the present disclosure, there is provided a method for preparing mesenchymal stem cells suspension from a clotted bone marrow, said method comprising: (1) obtaining a bone marrow sample, wherein the bone marrow sample comprises clotted bone marrow; (2) chopping the clotted bone marrow into pieces of at least 2 mm³ to obtain chopped clotted bone marrow; (3) contacting the chopped clotted bone marrow to at least one or combinations of enzymes in presence of a buffer to obtain a clotted bone marrow reaction solution; (4) incubating the clotted bone marrow reaction solution at a temperature in a range of 35° C. to 39° C. for a time of at least 20 minutes at a speed in a range of 100 rpm to 200 rpm to obtain incubated clotted bone marrow reaction solution; (5) contacting the incubated clotted bone marrow reaction solution of step (4) to a growth medium to obtain a suspension, wherein the growth medium is selected from a group consisting of α-MEM, DMEM, IMDM, F12, EMEM and combinations thereof; (6) mixing the suspension for a plurality of repeats; (7) filtering the suspension of step (6) with a cell strainer with a pore size in a range of 50 μm-100 μm to obtain a filtrate; (8) centrifuging the filtrate at 1300 rpm to 1800 rpm for a time in a range of 10 minutes to 15 minutes to obtain a cell pellet; and (9) dissolving the cell pellet with a nutrient medium to obtain a mesenchymal stem cell suspension, wherein the nutrient medium comprises 10% to 20% of a platelet lysate, and a medium selected from a group consisting of α-MEM, DMEM, IMDM, F12, EMEM and combinations thereof, and a plurality of factors selected from a group consisting of L-Glutamine, FGF, TGF, and wherein the platelet lysate comprises a lysate obtained from a mixture of an umbilical cord blood (UCB) derived platelet-rich plasma and a maternal blood (MB) derived platelet-rich plasma, and the mixture of an umbilical cord blood (UCB) derived platelet-rich plasma and a maternal blood (MB) derived platelet-rich plasma comprises 0.3×10⁹ to 1.5×10⁹ platelets/ml.

In an embodiment of the present disclosure, there is provided a method for preparing osteoblast cell suspension from mesenchymal stem cell suspension, said method comprising: (i) seeding in a culture flask, a mesenchymal stem cell suspension in a nutrient medium comprising 10% to 20% of a platelet lysate in the nutrient medium, to obtain culture flask-adhered mesenchymal stem cells; (ii) culturing the adhered mesenchymal stem cells in a nutrient medium comprising 10% to 20% of a platelet lysate in the nutrient medium; (iii) supplementing the nutrient medium of step (ii) with differentiation factors and growth factors to obtain a differentiation medium; (iv) complementing the differentiation nutrient medium of step (iii) with fresh differentiation nutrient medium comprising 10% to 20% of a platelet lysate, differentiation factors and growth factors to obtain a population of pre-osteoblast cells; (v) sub-culturing the population of pre-osteoblast cells of step (iv) for a time in a range of 12 to 15 days to obtain a P1 pre-osteoblast cells; (vi) expanding the P1 pre-osteoblast cells in a P1 expansion nutrient medium comprising 10% to 20% of a platelet lysate in the P1 expansion nutrient medium for a time in a range of 12 to 15 days to obtain expanded P1 pre-osteoblast cells, and sub-culturing the expanded P1 pre-osteoblast cells, to obtain a P2 osteoblast cells; and (vii) expanding the P2 osteoblast cells in a P2 expansion nutrient medium comprising 10% to 20% of a platelet lysate in the P2 expansion nutrient medium to obtain osteoblast cell suspension, wherein the mesenchymal stem cell suspension is prepared using a method as described herein, and wherein nutrient medium for culturing the adhered mesenchymal stem cells comprises a medium selected from a group consisting of α-MEM, DMEM, IMDM, F12, EMEM and combinations thereof; and a plurality of factors selected from a group consisting of L-glutamine, FGF, TGF, and wherein the differentiation medium comprises a medium selected from a group consisting of α-MEM, DMEM, IMDM, F12, EMEM and combinations thereof; and a plurality of factors selected from a group consisting of L-glutamine, FGF, TGF, IGF, L-thyroxine, calcitrol, stanozolol, dexamethasone, and wherein the P1 expansion medium comprises a medium selected from a group consisting of α-MEM, DMEM, IMDM, F12, EMEM and combinations thereof; and a plurality of factors selected from a group consisting of L-glutamine, F12, genistein, FGF, TGF, IGF, and wherein the P2 expansion medium comprises a medium selected from a group consisting of α-MEM, DMEM, IMDM, F12, EMEM and combinations thereof; and a plurality of factors selected from a group consisting of L-glutamine, F12, genistein, FGF, TGF, IGF.

In an embodiment of the present disclosure, there is provided a method for preparing osteoblast cell suspension from mesenchymal stem cell suspension, said method comprising: (i) seeding in a culture flask, a mesenchymal stem cell suspension in a nutrient medium comprising 10% to 20% of a platelet lysate in the nutrient medium, to obtain culture flask-adhered mesenchymal stem cells; (ii) culturing the adhered mesenchymal stem cells in a nutrient medium comprising 10% to 20% of a platelet lysate in the nutrient medium; (iii) supplementing the nutrient medium of step (ii) with differentiation factors and growth factors to obtain a differentiation medium; (iv) complementing the differentiation nutrient medium of step (iii) with fresh differentiation nutrient medium comprising 10% to 20% of a platelet lysate, differentiation factors and growth factors to obtain a population of pre-osteoblast cells; (v) sub-culturing the population of pre-osteoblast cells of step (iv) for a time in a range of 12 to 15 days to obtain a P1 pre-osteoblast cells; (vi) expanding the P1 pre-osteoblast cells in a P1 expansion nutrient medium comprising 10% to 20% of a platelet lysate in the P1 expansion nutrient medium for a time in a range of 12 to 15 days to obtain expanded P1 pre-osteoblast cells, and sub-culturing the expanded P1 pre-osteoblast cells, to obtain a P2 osteoblast cells; and (vii) expanding the P2 osteoblast cells in a P2 expansion nutrient medium comprising 10% to 20% of a platelet lysate in the P2 expansion nutrient medium to obtain osteoblast cell suspension, wherein the mesenchymal stem cell suspension is prepared using a method as described herein, and wherein the mesenchymal stem cells are culture flask-adherent, nucleated, and stain with crystal violet stain, and wherein the mesenchymal stem cells test positive in flow cytometry cell surface marker analysis for CD90, CD73 and CD105, and negative for CD34 and HLA-DR, and wherein the mesenchymal stem cells test positive for OCT-4, Nanog, and Sox-2 markers in RT-PCR analysis, and wherein the P1 pre-osteoblast cells test positive in flow cytometry cell surface marker analysis for bone ALP, and wherein the P1 pre-osteoblast cells test positive for ALP, collagen-1, osterix, and runx2 and negative for ephrinB4 markers in RT-PCR analysis, and wherein the P1 pre-osteoblast cells test positive for bone ALP by histochemical analysis, and wherein the P2 osteoblast cells test positive in flow cytometry cell surface marker analysis for ALP, and the P2 osteoblast cells test positive for alizarin red staining, and wherein the P2 osteoblast cells test positive for bone ALP, collagen-1, osterix, runx2 and ephrinB4 markers in RT-PCR analysis, and wherein the osteoblast cells are in a range of 12×10⁶ cells to 60×10⁶ cells.

In an embodiment of the present disclosure, there is provided a method for preparing osteoblast cell suspension from mesenchymal stem cell suspension, said method comprising: (i) seeding in a culture flask, a mesenchymal stem cell suspension in a nutrient medium comprising 10% to 20% of a platelet lysate in the nutrient medium, to obtain culture flask-adhered mesenchymal stem cells; (ii) culturing the adhered mesenchymal stem cells in a nutrient medium comprising 10% to 20% of a platelet lysate in the nutrient medium; (iii) supplementing the nutrient medium of step (ii) with differentiation factors and growth factors to obtain a differentiation medium; (iv) complementing the differentiation nutrient medium of step (iii) with fresh differentiation nutrient medium comprising 10% to 20% of a platelet lysate, differentiation factors and growth factors to obtain a population of pre-osteoblast cells; (v) sub-culturing the population of pre-osteoblast cells of step (iv) for a time in a range of 12 to 15 days to obtain a P1 pre-osteoblast cells; (vi) expanding the P1 pre-osteoblast cells in a P1 expansion nutrient medium comprising 10% to 20% of a platelet lysate in the P1 expansion nutrient medium for a time in a range of 12 to 15 days to obtain expanded P1 pre-osteoblast cells, and sub-culturing the expanded P1 pre-osteoblast cells, to obtain a P2 osteoblast cells; and (vii) expanding the P2 osteoblast cells in a P2 expansion nutrient medium comprising 10% to 20% of a platelet lysate in the P2 expansion nutrient medium to obtain osteoblast cell suspension, wherein the platelet lysate comprises a lysate obtained from a mixture of an umbilical cord blood (UCB) derived platelet-rich plasma and a maternal blood (MB) derived platelet-rich plasma, and wherein the mixture of an umbilical cord blood (UCB) derived platelet-rich plasma and a maternal blood (MB) derived platelet-rich plasma comprises 0.3×10⁹ to 1.5×10⁹ platelets/ml. In another embodiment of the present disclosure, the mixture of an umbilical cord blood (UCB) derived platelet-rich plasma and a maternal blood (MB) derived platelet-rich plasma comprises 0.3×10⁹ to 0.7×10⁹ platelets/ml. In yet another embodiment of the present disclosure, the mixture of an umbilical cord blood (UCB) derived platelet-rich plasma and a maternal blood (MB) derived platelet-rich plasma comprises 0.3×10⁹ to 0.5×10⁹ platelets/ml.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; and (g) delivering the osteoblast cell-mixture to a site in a subject. In another embodiment there is provided a method of delivering osteoblast cells into a subject as described herein, the osteoblast cell suspension is prepared from a mesenchymal stem cell suspension, and said mesenchymal stem cell suspension is obtained from a clotted bone marrow sample.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; and (g) delivering the osteoblast cell-mixture to a site in a subject, wherein the osteoblast cell suspension is prepared from a mesenchymal stem cell suspension, and said mesenchymal stem cell suspension is obtained from a clotted bone marrow sample, and wherein the mesenchymal stem cell suspension is cultured in presence of a nutrient medium comprising a platelet lysate to obtain the osteoblast cell suspension, and the platelet lysate comprises a lysate obtained from a mixture of an umbilical cord blood (UCB) derived platelet rich plasma and a maternal blood (MB) derived platelet rich plasma, and the mixture of an umbilical cord blood (UCB) derived platelet rich plasma and a maternal blood (MB) derived platelet rich plasma comprises 0.3×10⁹ to 1.5×10⁹ platelets/ml.

In an embodiment of the present disclosure, there is provided a method of delivering osteoblast cells into a subject as described herein, wherein the first mixture comprises 100-600 IU/ml thrombin, or 200-550 IU/ml thrombin. In another embodiment of the present disclosure, there is provided a method of delivering osteoblast cells into a subject as described herein, the third mixture comprises 20-100 mg/ml of fibrinogen, or 25-70 mg/ml of fibrinogen.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; and (g) delivering the osteoblast cell-mixture to a site in a subject, wherein the first nutrient medium of step (b) or the second nutrient medium of step (e) comprises at least one medium selected from a group consisting of DMEM, αMEM, IMDM, and combinations thereof.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; and (g) delivering the osteoblast cell-mixture to a site in a subject, wherein the osteoblast cell suspension comprises osteoblast cells in a range of 12×10⁶ cells to 60×10⁶ cells. In another embodiment of the present disclosure, the osteoblast cell suspension comprises osteoblast cells in a range of 24×10⁶ cells to 55×10⁶ cells. In yet another embodiment, the osteoblast cell suspension comprises osteoblast cells in a range of 40×10⁶ cells to 55×10⁶ cells.

In an embodiment of the present disclosure, there is provided a method of delivering osteoblast cells into a subject as described herein, wherein the mixing of the second mixture and the third mixture, and the delivering of the osteoblast cell suspension is done by a dual syringe device.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; and (g) delivering the osteoblast cell-mixture to a site in a subject, wherein the second mixture and the third mixture is mixed in a ratio having a range of 1:0.5 to 1:2. In another embodiment of the present disclosure, the second mixture and the third mixture is mixed in a ratio having a range of 1:0.8 to 1:1.5. In yet another embodiment of the present disclosure, the second mixture and the third mixture is mixed in a ratio of 1:1.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; and (g) delivering the osteoblast cell-mixture to a site in a subject, wherein the method achieves in-vivo bone regeneration.

In an embodiment of the present disclosure, there is provided a method of delivering osteoblast cells into a subject as described herein, wherein fibrinogen is obtained from a mixture of umbilical cord blood (UCB) plasma and maternal blood (MB) plasma. In another embodiment of the present disclosure, there is provided a method of delivering osteoblast cells into a subject as described herein thrombin is obtained from a mixture of umbilical cord blood (UCB) plasma and maternal blood (MB) plasma.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; and (g) delivering the osteoblast cell-mixture to a site in a subject, wherein the osteoblast cell-mixture forms a gel network in a time in a range of 3-6 minutes.

In an embodiment of the present disclosure, there is provided a method of delivering osteoblast cells into a subject as described herein, wherein the osteoblast cell-mixture comprises autologous osteoblast cells.

In an embodiment of the present disclosure, there is provided a method of delivering osteoblast cells into a subject as described herein, wherein the method is used to correct conditions selected from a group consisting of non-union fracture, fibrous dysplasia, avascular necrosis, oral and maxillofacial fractures, and sinus lift.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; and (g) delivering the osteoblast cell-mixture to a site in a subject, wherein thrombin is obtained by a method comprising: (i) mixing umbilical cord blood plasma with maternal blood plasma to obtain mixture A; (ii) contacting saturated ammonium sulphate/ethanol to the mixture A in a ratio selected from a group consisting of 1:1, 2:1 and 3:1, wherein the ratio is between cord blood in mixture A and the saturated ammonium sulphate/ethanol, and allowed to precipitate plasma proteins for 5-20 minutes to obtain a mixture B; (iii) centrifuging the mixture B at 3000-5000 rpm for 5-10 minutes to separate protein precipitate in form of a pellet from a supernatant; (iv) discarding the supernatant and adding 4-5 ml of a solution A to the pellet to obtain a pellet solution; (v) centrifuging the pellet solution, and collecting supernatant; (vi) lyophilizing the supernatant under vacuum conditions to obtain a lyophilized protein powder; (vii) dissolving the lyophilized protein powder in a solution B to obtain a protein solution; (viii) extracting prothrombin by ion exchange chromatography using diethyl aminoethyl (DEAE-IEC) followed by heparin affinity chromatography (A second DEAE-IEC step), followed by immobilized metal affinity chromatography (IMAC), and collecting prothrombin; (ix) activating prothrombin obtain crude thrombin and purifying crude thrombin by hydrophobic interaction chromatography (HIC), to obtain purified thrombin; (x) concentrating the purified thrombin, to obtain thrombin, wherein the solution A and solution B is independently selected from a group consisting of water, saline, and Dulbecco's phosphate buffer saline. In another embodiment of the present disclosure, there is provided a method of delivering osteoblast cells into a subject as described herein, wherein fibrinogen is obtained by a method comprising: (i) mixing umbilical cord blood plasma with maternal blood plasma to obtain mixture A; (ii) contacting saturated ammonium sulphate/ethanol to the mixture A in a ratio selected from a group consisting of 1:1, 2:1 and 3:1, wherein the ratio is between cord blood in mixture A and the saturated ammonium sulphate/ethanol, and allowed to precipitate plasma proteins for 5-20 minutes to obtain a mixture B; (iii) centrifuging the mixture B at 3000-5000 rpm for 5-10 minutes to separate a pellet from a supernatant; (iv) lyophilizing the supernatant under vacuum conditions resulting in a lyophilized protein powder, wherein the lyophilized protein powder comprises fibrinogen wherein the solution A is selected from a group consisting of water, saline, and Dulbecco's phosphate buffer saline.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) adjusting position of the subject in gravity-dependent position; (j) injecting the osteoblast cell-mixture of step (f) into the defect area and allowing the cell mixture to form a gel network; and (k) applying standard medical procedures for suturing, to deliver osteoblast cells into the subject.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) performing curettage to clean necrosed bone present in the defect area; (j) washing the holes by a saline solution; (k) adjusting position of the subject in a gravity-dependent position; (l) injecting the osteoblast cell-mixture of step (f) into the defect area and allowing the cell mixture to form a gel network; and (m) applying standard medical procedures for suturing, to deliver osteoblast cells into the subject.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) adjusting position of the subject in gravity-dependent position; (j) injecting the osteoblast cell-mixture of step (f) into the defect area and allowing the cell mixture to form a gel network; and (k) applying standard medical procedures for suturing, to deliver osteoblast cells into the subject, wherein the osteoblast cell suspension is prepared from a mesenchymal stem cell suspension, and said mesenchymal stem cell suspension is obtained from a clotted bone marrow sample.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) adjusting position of the subject in gravity-dependent position; (j) injecting the osteoblast cell-mixture of step (f) into the defect area and allowing the cell mixture to form a gel network; and (k) applying standard medical procedures for suturing, to deliver osteoblast cells into the subject, wherein the osteoblast cell suspension is prepared from a mesenchymal stem cell suspension, and said mesenchymal stem cell suspension is obtained from a clotted bone marrow sample, and wherein the mesenchymal stem cell suspension is cultured in presence of a nutrient medium comprising a platelet lysate to obtain the osteoblast cell suspension, and the platelet lysate comprises a lysate obtained from a mixture of an umbilical cord blood (UCB) derived platelet rich plasma and a maternal blood (MB) derived platelet rich plasma, and mixture of an umbilical cord blood (UCB) derived platelet rich plasma and a maternal blood (MB) derived platelet rich plasma comprises 0.3×10⁹ to 1.5×10⁹ platelets/ml.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) adjusting position of the subject in gravity-dependent position; (j) injecting the osteoblast cell-mixture of step (f) into the defect area and allowing the cell mixture to form a gel network; and (k) applying standard medical procedures for suturing, to deliver osteoblast cells into the subject, wherein the first mixture comprises 100-600 IU/ml thrombin. In another embodiment of the present disclosure, the first mixture comprises 200-600 IU/ml thrombin. In yet another embodiment, the first mixture comprises 300-550 IU/ml thrombin

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) adjusting position of the subject in gravity-dependent position; (j) injecting the osteoblast cell-mixture of step (f) into the defect area and allowing the cell mixture to form a gel network; and (k) applying standard medical procedures for suturing, to deliver osteoblast cells into the subject, wherein the third mixture comprises 20-100 mg/ml of fibrinogen. In another embodiment, the third mixture comprises 50-100 mg/ml of fibrinogen.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) adjusting position of the subject in gravity-dependent position; (j) injecting the osteoblast cell-mixture of step (f) into the defect area and allowing the cell mixture to form a gel network; and (k) applying standard medical procedures for suturing, to deliver osteoblast cells into the subject, wherein the first nutrient medium of step (b) or the second nutrient medium of step (e) comprises at least one medium selected from a group consisting of DMEM, αMEM, IMDM, and combinations thereof.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) adjusting position of the subject in gravity-dependent position; (j) injecting the osteoblast cell-mixture of step (f) into the defect area and allowing the cell mixture to form a gel network; and (k) applying standard medical procedures for suturing, to deliver osteoblast cells into the subject, wherein the osteoblast cell suspension comprises osteoblast cells in a range of 12×10⁶ cells to 60×10⁶ cells. In another embodiment of the present disclosure, the osteoblast cell suspension comprises osteoblast cells in a range of 24×10⁶ cells to 55×10⁶ cells. In yet another embodiment, the osteoblast cell suspension comprises osteoblast cells in a range of 40×10⁶ cells to 52×10⁶ cells.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) adjusting position of the subject in gravity-dependent position; (j) injecting the osteoblast cell-mixture of step (f) into the defect area and allowing the cell mixture to form a gel network; and (k) applying standard medical procedures for suturing, to deliver osteoblast cells into the subject, wherein the injecting of the osteoblast cell-mixture is done by a dual syringe device.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) adjusting position of the subject in gravity-dependent position; (j) injecting the osteoblast cell-mixture of step (f) into the defect area and allowing the cell mixture to form a gel network; and (k) applying standard medical procedures for suturing, to deliver osteoblast cells into the subject, wherein the second mixture and the third mixture is mixed in a ratio having a range of 1:0.5 to 1:2. In another embodiment of the present disclosure, the second mixture and the third mixture is mixed in a ratio of 1:1.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) adjusting position of the subject in gravity-dependent position; (j) injecting the osteoblast cell-mixture of step (f) into the defect area and allowing the cell mixture to form a gel network; and (k) applying standard medical procedures for suturing, to deliver osteoblast cells into the subject, wherein the method achieves in-vivo bone regeneration.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) adjusting position of the subject in gravity-dependent position; (j) injecting the osteoblast cell-mixture of step (f) into the defect area and allowing the cell mixture to form a gel network; and (k) applying standard medical procedures for suturing, to deliver osteoblast cells into the subject, wherein fibrinogen is obtained from a mixture of umbilical cord blood (UCB) plasma and maternal blood (MB) plasma. In another embodiment of the present disclosure, there is provided a method of delivering osteoblast cells into a subject as described herein, wherein thrombin is obtained from a mixture of umbilical cord blood (UCB) plasma and maternal blood (MB) plasma.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) adjusting position of the subject in gravity-dependent position; (j) injecting the osteoblast cell-mixture of step (f) into the defect area and allowing the osteoblast cell-mixture to form a gel network; and (k) applying standard medical procedures for suturing, to deliver osteoblast cell-mixture into the subject, wherein the gel network is formed in a time in a range of 3-6 minutes.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) adjusting position of the subject in gravity-dependent position; (j) injecting the osteoblast cell-mixture of step (f) into the defect area and allowing the cell mixture to form a gel network; and (k) applying standard medical procedures for suturing, to deliver osteoblast cells into the subject, wherein the osteoblast cell-mixture comprises autologous osteoblast cells. In another embodiment of the present disclosure, the osteoblast cell-mixture comprises allogenic osteoblast cells.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) adjusting position of the subject in gravity-dependent position; (j) injecting the osteoblast cell-mixture of step (f) into the defect area and allowing the cell mixture to form a gel network; and (k) applying standard medical procedures for suturing, to deliver osteoblast cells into the subject, wherein the method is used to correct conditions selected from a group consisting of non-union fracture, fibrous dysplasia, avascular necrosis, oral and maxillofacial fractures, and sinus lift.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) adjusting position of the subject in gravity-dependent position; (j) injecting the osteoblast cell-mixture of step (f) into the defect area and allowing the cell mixture to form a gel network; and (k) applying standard medical procedures for suturing, to deliver osteoblast cells into the subject, wherein drilling holes is through a 7/9 mm sized reamer made by standard core decompression method to approach a necrotic area, and the necrotic area is debrided with the help of long surgical scoops to gain access to a defect area, and wherein in the defect area, long spinal needle is inserted in core decompression tunnel and tip is placed at the defect area region under C-Arm guidance.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) adjusting position of the subject in gravity-dependent position; (j) injecting the osteoblast cell-mixture of step (f) into the defect area and allowing the cell mixture to form a gel network; and (k) applying standard medical procedures for suturing, to deliver osteoblast cells into the subject, wherein thrombin is obtained by a method comprising: (i) mixing umbilical cord blood plasma with maternal blood plasma to obtain mixture A; (ii) contacting saturated ammonium sulphate/ethanol to the mixture A in a ratio selected from a group consisting of 1:1, 2:1 and 3:1, wherein the ratio is between cord blood in mixture A and the saturated ammonium sulphate/ethanol, and allowed to precipitate plasma proteins for 5-20 minutes to obtain a mixture B; (iii) centrifuging the mixture B at 3000-5000 rpm for 5-10 minutes to separate protein precipitate in form of a pellet from a supernatant; (iv) discarding the supernatant and adding 4-5 ml of a solution A to the pellet to obtain a pellet solution; (v) centrifuging the pellet solution, and collecting supernatant; (vi) lyophilizing the supernatant under vacuum conditions to obtain a lyophilized protein powder; (vii) dissolving the lyophilized protein powder in a solution B to obtain a protein solution; (viii) extracting prothrombin by ion exchange chromatography using diethyl aminoethyl (DEAE-IEC) followed by heparin affinity chromatography (A second DEAE-IEC step), followed by immobilized metal affinity chromatography (IMAC), and collecting prothrombin; (ix) activating prothrombin obtain crude thrombin and purifying crude thrombin by hydrophobic interaction chromatography (HIC), to obtain purified thrombin; (x) concentrating the purified thrombin, to obtain thrombin, wherein the solution A and solution B is independently selected from a group consisting of water, saline, and Dulbecco's phosphate buffer saline.

In an embodiment of the present disclosure, there is provided a method of delivering osteoblast cells into a subject, said method comprising: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; (f) mixing the second mixture of step (c) with the third mixture of step (e), to obtain an osteoblast cell-mixture; (g) incising lateral side of an affected area in a subject to create an incision; (h) drilling holes through the incision to gain access to a defect area; (i) adjusting position of the subject in gravity-dependent position; (j) injecting the osteoblast cell-mixture of step (f) into the defect area and allowing the cell mixture to form a gel network; and (k) applying standard medical procedures for suturing, to deliver osteoblast cells into the subject, wherein fibrinogen is obtained by a method comprising: (i) mixing umbilical cord blood plasma with maternal blood plasma to obtain mixture A; (ii) contacting saturated ammonium sulphate/ethanol to the mixture A in a ratio selected from a group consisting of 1:1, 2:1 and 3:1, wherein the ratio is between cord blood in mixture A and the saturated ammonium sulphate/ethanol, and allowed to precipitate plasma proteins for 5-20 minutes to obtain a mixture B; (iii) centrifuging the mixture B at 3000-5000 rpm for 5-10 minutes to separate a pellet from a supernatant; (iv) lyophilizing the supernatant under vacuum conditions resulting in a lyophilized protein powder, wherein the lyophilized protein powder comprises fibrinogen wherein the solution A is selected from a group consisting of water, saline, and Dulbecco's phosphate buffer saline.

In an embodiment of the present disclosure, there is provided a method for preparing an osteoblast cell-mixture, said method comprising: (a) obtaining umbilical cord blood (UCB) derived platelet rich plasma; (b) obtaining maternal blood (MB) derived platelet rich plasma; (c) contacting umbilical cord blood (UCB) derived platelet rich plasma and maternal blood (MB) derived platelet rich plasma in a ratio range of 10:1 to 30:1, to obtain a platelet rich plasma (PRP) mix; (d) obtaining at least one activator; and (e) contacting the PRP mix, and the at least one activator with an osteoblast cell suspension, to obtain the osteoblast cell-mixture. In another embodiment, contacting umbilical cord blood (UCB) derived platelet rich plasma and maternal blood (MB) derived platelet rich plasma is done in a ratio range of 15:1 to 25:1. In yet another embodiment, the ratio is 18:1 to 22:1.

In an embodiment of the present disclosure, there is provided a method for preparing an osteoblast cell-mixture as described herein, wherein the at least one activator is selected from a group consisting of calcium gluconate, calcium saccharate, deoxy gluconate, calcium glucoheptonate, calcium chloride, and combinations thereof.

In an embodiment of the present disclosure, there is provided a method of delivering osteoblast cells into a subject, said method comprising: (a) obtaining umbilical cord blood (UCB) derived platelet rich plasma; (b) obtaining maternal blood (MB) derived platelet rich plasma; (c) contacting umbilical cord blood (UCB) derived platelet rich plasma and maternal blood (MB) derived platelet rich plasma in a ratio range of 10:1 to 30:1, to obtain a platelet rich plasma (PRP) mix; (d) obtaining at least one activator; (e) contacting the PRP mix, and the at least one activator with an osteoblast cell suspension, to obtain an osteoblast cell-mixture; and (f) delivering the osteoblast cell-mixture to a site in a subject.

In an embodiment of the present disclosure, there is provided a method of delivering osteoblast cells into a subject, said method comprising: (a) obtaining umbilical cord blood (UCB) derived platelet rich plasma; (b) obtaining maternal blood (MB) derived platelet rich plasma; (c) contacting umbilical cord blood (UCB) derived platelet rich plasma and maternal blood (MB) derived platelet rich plasma in a ratio range of 10:1 to 30:1, to obtain a platelet rich plasma (PRP) mix; (d) obtaining at least one activator; (e) contacting the PRP mix, and the at least one activator with an osteoblast cell suspension, to obtain an osteoblast cell-mixture; (f) incising lateral side of an affected area in a subject to create an incision; (g) drilling holes through the incision to gain access to a defect area; (h) adjusting position of the subject in gravity-dependent position; (i) injecting the osteoblast cell-mixture of step (e) into the defect area and allowing the cell mixture to form a gel network; and (j) applying standard medical procedures for suturing, to deliver osteoblast cell-mixture into the subject.

In an embodiment of the present disclosure, there is provided a method for preparing an osteoblast cell-mixture, said method comprising: (a) obtaining a platelet lysate comprising a lysate obtained from a mixture of an umbilical cord blood (UCB) derived platelet rich plasma and a maternal blood (MB) derived platelet rich plasma; (b) obtaining at least one activator; and (c) contacting the platelet lysate and the at least one activator with an osteoblast cell suspension, to obtain the osteoblast cell-mixture.

In an embodiment of the present disclosure, there is provided a method of delivering osteoblast cells into a subject, said method comprising: (a) obtaining a platelet lysate comprising a lysate obtained from a mixture of an umbilical cord blood (UCB) derived platelet rich plasma and a maternal blood (MB) derived platelet rich plasma; (b) obtaining at least one activator; (c) contacting the platelet lysate and the at least one activator with an osteoblast cell suspension, to obtain the osteoblast cell-mixture; and (d) delivering the osteoblast cell-mixture to a site in a subject.

In an embodiment of the present disclosure, there is provided a method of delivering osteoblast cells into a subject, said method comprising: (a) obtaining a platelet lysate comprising a lysate obtained from a mixture of an umbilical cord blood (UCB) derived platelet rich plasma and a maternal blood (MB) derived platelet rich plasma; (b) obtaining at least one activator; (c) contacting the platelet lysate and the at least one activator with an osteoblast cell suspension, to obtain the osteoblast cell-mixture; (d) incising lateral side of an affected area in a subject to create an incision; (e) drilling holes through the incision to gain access to a defect area; (f) adjusting position of the subject in gravity-dependent position; (g) injecting the osteoblast cell-mixture of step (c) into the defect area and allowing the cell mixture to form a gel network; and (h) applying standard medical procedures for suturing, to deliver osteoblast cell-mixture into the subject.

Although the subject matter has been described in considerable detail with reference to certain examples and implementations thereof, other implementations are possible.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein. It is to be understood that this disclosure is not limited to particular methods, and experimental conditions described, as such methods and conditions may apply.

The examples as described herein clearly illustrates the process followed for arriving at the present disclosure. The experimental efforts have been bought out in the present example section.

### Example 1

Preparation of Osteoblast Cells from Mesenchymal Stem Cells: Covering the Process of Obtaining MSC from Bone Marrow and Culturing the MSC to Obtain Osteoblast Cells

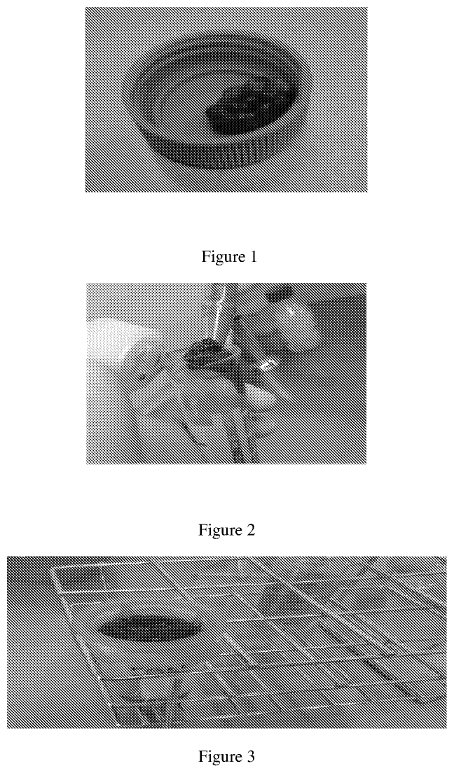

Isolating MSC from clotted bone marrow sample: The bone marrow biopsy was collected in a transport vial containing transport medium (90% α-MEM+10% platelet lysate (PL)). The PL as described in the present and following examples is the platelet lysate comprising a lysate obtained from a mixture of an umbilical cord blood (UCB) derived platelet rich plasma and a maternal blood (MB) derived platelet rich plasma, wherein the mixture of an umbilical cord blood (UCB) derived platelet rich plasma and a maternal blood (MB) derived platelet rich plasma comprises 0.3×10⁹ to 1.5×10⁹ platelets/ml. The clotted bone marrow sample (FIG. 1 and FIG. 2) was not suitable for isolation of osteoprogenitor cells/MSCs and might result in discard of the bone marrow sample. To overcome this problem, clotted bone marrow sample was placed on 100 μm cell strainer (FIG. 3) on top of a sterile 50 ml centrifuge tube. Bone marrow was transferred and aspirated from the transport kit onto the cell strainer. Clotted bone marrow collected on cell strainer taken into an empty cell culture Petri dish using sterile forceps. Clot was chopped into small pieces of approximately 2-3 mm³ using a sterile scalpel and sterile needle. Small pieces of the clot transferred into a 50 ml centrifuge tube using the sterile forceps.

To these small chopped pieces of bone clots, 10 ml of freshly prepared enzymes (Urokinase mixed with enzymes including but not limiting to collagenase enzymes (either Type I or Type II) and Hyaluronidase, and heat shock protein 70 (HSP 70), suspended in Hank's balanced salt solution (1×-HBSS)) was added to the clot pieces and incubated for 20-30 min at 37° C. in a shaking incubator at 150 rpm. Further, 10 ml of growth medium in the clotted biopsy-enzyme solution was added and vigorous mixing of the suspension was done using 25 ml sterile pipette. The above vigorous mixing procedure was repeated for 3-4 times. Growth medium containing bone marrow sample was filtered with 50-100 μM cell strainer followed by centrifugation at 1300-1800 rpm for 10-15 min. After centrifugation supernatant was discarded and cell pellet dissolved with growth medium (DMEM with 10% PL/FBS). Different tests viz., cell count, cell viability (Table-01) and other QC tests viz., sterility and mycoplasma were performed. 1 ml of each nucleated cells of bone marrow cell suspension were seeded in four 35 mm Petri plates for confirmation of CFU test and cell characterization through flow cytometry. Remaining cell suspension was seeded in T₇₅ tissue culture grade cell culture flasks for further culture, differentiation into osteoblasts and their expansion with different combination of medium viz., DMEM, F12, RPMI and α-MEM along with 10% umbilical cord blood derived Platelet lysate with maternal platelet lysate—PL with growth factor FGF. Ten different clotted bone marrow samples were used to isolate MSCs, the isolated MSCs were further differentiated to osteoblast cells. Table 1 provides MSC characterization data for samples 1-10. The sample numbers refer to the respective sample as per the context (MSCs at isolation stage, or osteoblast at differentiation/expansion stage). The procedure is the same to be followed for the un-clotted bone marrow or normal bone marrow.

Data with respect to cell count, viability, and sterility for ten samples which were isolated from clotted bone marrow have been described in Table 1 which are cultured using the UCB+MB PL as described herein.

TABLE 1

| | | | Cell | | |
|---|---|---|---|---|---|
| | | | viability at | | |
| | | Cell count at | isolation | | |
| S. No | Sample ID | isolation stage | stage | Sterility | Mycoplasma |
| 1 | Sample-01 | 5.1 × 10⁶ | 98.2 | No growth | Negative |
| 2 | Sample-02 | 4.4 × 10⁶ | 97.5 | No growth | Negative |
| 3 | Sample-03 | 3.6 × 10⁶ | 96.4 | No growth | Negative |
| 4 | Sample-04 | 4.8 × 10⁶ | 95.7 | No growth | Negative |
| 5 | Sample-05 | 5.6 × 10⁶ | 90.8 | No growth | Negative |
| 6 | Sample-06 | 5.2 × 10⁶ | 89.6 | No growth | Negative |
| 7 | Sample-07 | 5.9 × 10⁶ | 93.8 | No growth | Negative |
| 8 | Sample-08 | 6.4 × 10⁶ | 97.5 | No growth | Negative |
| 9 | Sample-09 | 4.9 × 10⁶ | 92.2 | No growth | Negative |
| 10 | Sample-10 | 5.3 × 10⁶ | 90.8 | No growth | Negative |
| 11 | Sample-11 | 6.2 × 10⁶ | 91.6 | No growth | Negative |
| 12 | Sample-12 | 6.4 × 10⁶ | 94.5 | No growth | Negative |
| 13 | Sample-13 | 3.9 × 10⁶ | 90.8 | No growth | Negative |
| 14 | Sample-14 | 4.6 × 10⁶ | 89.9 | No growth | Negative |
| 15 | Sample-15 | 4.1 × 10⁶ | 96.6 | No growth | Negative |

The data provided in Table 1 confirms the isolation of MSC from bone marrow with good recovery.

### Characterization of MSC by Flow Cytometry and MSC-CFU Assay

The cells were seeded for MSC-CFU assay and flow cytometry for initial cell characterization in four 90 mm cell culture Petri plates, are used. Bone marrow samples (2 ml each) were seeded in 90 mm Petri plate cultured with Alpha MEM and 10% PL or FBS, 2 mM L-Glutamine and 1× pen-strep antibiotics (growth medium) along with growth factor such as 1-10 ng/ml FGF. These Petri plates were further incubated in CO₂ incubator with 5% CO₂ at 37° C. temperature conditions.

Medium changes (α-MEM and 10% PL or FBS) were given at subsequence intervals of 2-4 days up to 12-14 days in order to replenish the media which gets depleted during cell growth and multiplication; which otherwise causes depletion of media or acidic nature to the media leading to improper growth. Therefore, media is changed at regular intervals.

### MSC-CFU Assay:

### Identification of MSCs Using CFU-F Assay:

CFU-F assay was used to enumerate or identify the number of MSCs within a given heterogeneous population of cells isolated from bone marrow. It was carried out by using freshly isolated primary cells from bone marrow.

### Use of Crystal Violet Staining for CFU-F Assay:

### Preparation of Early Passage MSCs for the CFU-F Assay:

Harvested bone marrow derived primary cells were seeded in 90 mm Petri plate with growth medium as described above. Medium Changes were given at alternate day. Cells were incubated in CO₂ incubator at 37° C. at 5% under humidified conditions. On day 14±3, cultured cells were taken out from incubator and observed under microscope for growth of the cells. After confirmation of growth, the cells were analyzed for crystal violet staining.

### Procedure for Staining the CFU-F:

After 14±3 days of culture, petri plates were removed from the CO₂ incubator, cell culture medium was pipetted out and cells were washed with buffer for three times. Cells were stained with 0.5% of Crystal violet solution freshly prepared in methanol and incubated at room temperature for 30 minutes. Stain was discarded and then plate washed with buffer followed by air drying.

### Counting Colonies:

Stained cells were observed under inverted microscope. Inverted 90 mm dish and score bottom of dish into four equal quadrants. 90 mm dish was placed, inverted onto the stage of a dissection microscope. Colonies were enumerated of each plate. By definition, a colony was having a minimum of 50 cells to be enumerated. After counting the colonies photographs of the same were taken under 100× magnification.

### Results: MSC CFU-F Assay:

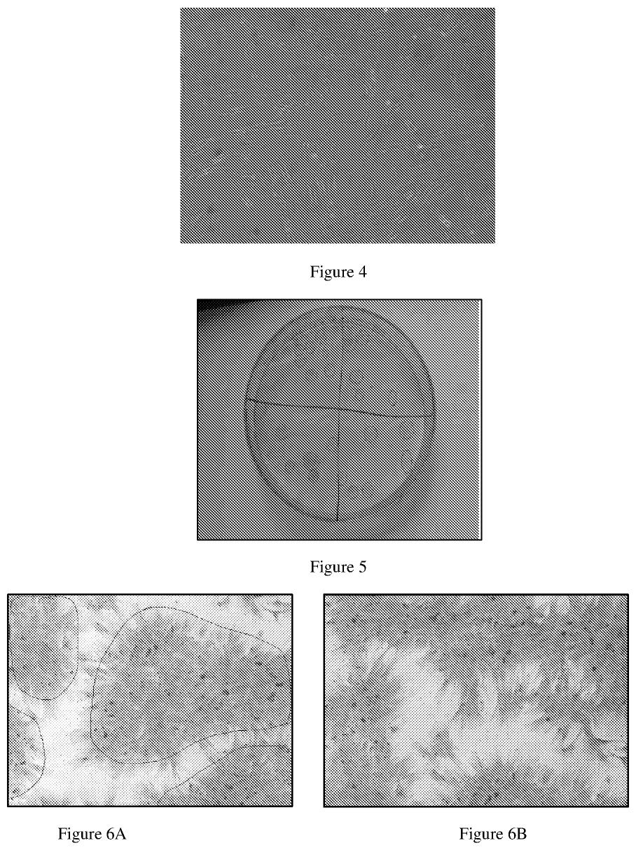

Table 2 depicts the results of Crystal violet in the stained plate containing BM-MSCs colonies at 14±3 days of culture. FIG. 4 depicts the BM-MSCs before straining. FIG. 5 depicts the Crystal violet staining showing stained BM-MSCs colonies in petri plates. FIG. 6 depicts the BM-MSC derived CFU-F colonies from clotted bone marrow at 100× magnification after staining. Therefore, Table 2 along with the FIGS. 3-6, suggest the staining of the cells, thereby signifying the isolation of MSC.

TABLE 2

| | | | | No. of |
|---|---|---|---|---|
| | | | | colonies |
| Sr. No | Sample ID | Result | Remark | obtained |
| 1 | Sample BM-MSC | Positive | Stained MSC | 25 |
| | 01 | | in colonies | |
| | | | were observed | |
| 2 | Sample BM-MSC | Positive | Stained MSC | 35 |
| | 02 | | in colonies | |
| | | | were observed | |
| 3 | Sample BM-MSC | Positive | Stained MSC | 45 |
| | 03 | | in colonies | |
| | | | were observed | |
| 4 | Sample BM-MSC | Positive | Stained MSC | 27 |
| | 04 | | in colonies | |
| | | | were observed | |
| 5 | Sample BM-MSC | Positive | Stained MSC | 24 |
| | 05 | | in colonies | |
| | | | were observed | |

### Characterization of BM-MSCs Using Flow Cytometry:

Around 1 million of cultured cell suspension was taken for cell characterization through flow cytometry. They were tested for CD90, CD73, and CD105 as a positive markers and CD 3 and HLA-DR as negative markers for expression on surface of BM-MSCs.

### Marker Characterization:

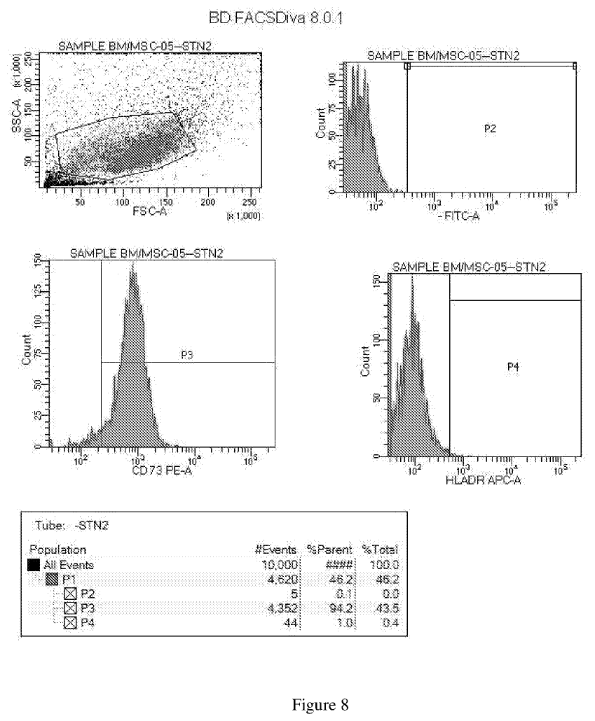

Cultured BMSCs (after 14±3 days) were harvested by the process of trypsinization. Immunophenotypic characterization was performed by Fluorescence-Activated Cell Sorter (FACS) on a BD (Becton, Dickinson) FACS CANTO-2. Fluorescence excitation was carried out by using an argon-ion and a red LASER of 488 nm and 632 nm respectively. The fluorescence emission was collected by using corresponding detectors. Approximately 1×10⁶ cells were stained with predefined antibody cocktails I i.e., CD 90, CD 73, CD 105, HLA-DR and CD 34. The stained cells were incubated in the dark for 20 min at room temperature, washed with FACS flow buffer (BD Biosciences) and resuspended in FACS flow buffer and then analyzed on a BD FACS canto-2. Data acquisition and analysis were accomplished by using BD FACS Diva software (BD Biosciences) (FIG. 8).

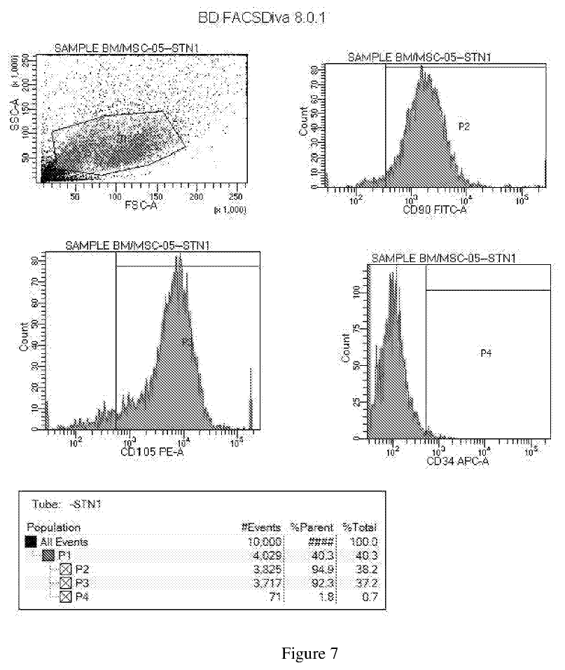

Table 3 depicts the cell surface marker expression for BM-MSCs. FIG. 7 depicts the FACS data for cell surface expression of different markers. Immunophenotypic results using Flow cytometry for bone marrow derived MSCs at 14±3 days of culture showed CD90 and CD105 positive expression and CD34 negative expression (Representation of Sample No. 05) (FIG. 7).

TABLE 3

| | | Cell Surface Expression (%) | | | | |
|---|---|---|---|---|---|---|
| Sr. No. | Sample ID | CD73 | CD90 | CD105 | HLA-DR | CD34 |
| 1 | Sample BM- | 99.8 | 99.9 | 100 | 0.1 | 3.6 |
| | MSC01 | | | | | |
| 2 | Sample BM- | 100 | 99.9 | 100 | 0.1 | 3.5 |
| | MSC 02 | | | | | |
| 3 | Sample BM- | 99.9 | 100 | 100 | 0.0 | 4.8 |
| | MSC 03 | | | | | |
| 4 | Sample BM- | 99.8 | 100 | 100 | 0.2 | 3.2 |
| | MSC 04 | | | | | |
| 5 | Sample BM- | 94.2 | 94.9 | 92.3 | 1.0 | 1.8 |
| | MSC 05 | | | | | |

Differentiation of BM-Derived MSC into Osteoblast Cells:

After 2-5 days of seeding of bone marrow nucleated cells, non-adhered cells are washed out during the process of medium change. To the adhered cells, bone differentiation medium with combinations of 60% α-MEM, 30% IMDM and L-glutamine (2 mM) with 10% umbilical cord blood derived platelet lysate with maternal blood platelet lysate—PL or FBS are used. Differentiation factors such as dexamethasone, β-glycerophosphate and L-ascorbic acid are added to the medium. Growth factors such as FGF, TGF, IGF were added to the differentiated medium. 10 ml of each freshly prepared medium are added to the culture flasks and flasks are further incubated in CO₂ incubator under humidified conditions (>80% humidity) with 5% CO₂ at 37° C. temperature conditions. The adhered cells at this stage, which is prior to expansion process have been termed as P0 (passage stage 0).

Expansion and Comparison with FBS or PL:

### Expansion:

After initiation of differentiation of MSCs into osteoblasts, media changes (EMEM+F12+FBS/PL) were given at subsequent intervals (every three days) to achieve >70% confluency stage, the cells at this stage has been termed as P1 (first passage stage) which took around 12-15 days. Cell were washed thrice with the 1× DPBS followed by treating with 0.25% trypsin and incubated the cells at 37° C. for 5 mins, followed by neutralizing the incubated cells with the complete media (EMEM+F12+FBS/PL). Cells were collected in growth media (EMEM+F12+FBS/PL) and centrifuged at 1300-1800 rpm for 5-10 mins. Cell count, cell viability (Table 4), sterility and mycoplasma (Table 5) were done subsequently. Cell characterization with the help of flow cytometry and RT-PCR studies are performed. For the cell characterization by flow cytometry alkaline phosphatase (ALP⁺) and for RT-PCR ALP, collagen-1, osterix and Runx markers are tested and rest of the cells were cryopreserved.

Culturing of Bone Cells Using FBS and Umbilical Cord Blood Derived Platelet Lysate (PL) with Maternal Blood Platelet Lysate:

In this study effect of platelet lysate on cell growth when added to medium and the same were compared with cell culture medium supplemented with FBS. Total Cell suspension was divided in two parts and these two parts cultured separately. One part contains a combination of DMEM, F12, RPMI and α-MEM with 10% umbilical cord blood derived platelet lysate with maternal blood platelet lysate and another containing cell suspension cultured in combination of DMEM, F12, RPMI and α-MEM with 10% FBS. In both the cases cells were seeded with seeding density of 3000 to 5000 cells/cm² in T-₁₅₀ cell culture flasks. Medium changes were given at subsequent intervals of 2-4 days to achieve >70% at second passage stage for around 12-15 days, cells at this stage have been termed as P2 (second passage stage). Cells were washed with the 1× DPBS followed by treating the rewashed cells with 0.25% trypsin and incubated the cells at 37° C. for 5 mins followed by neutralizing the incubated cells with the complete media. Cells were collected with growth media and centrifuged at 1300-1800 rpm for 5-10 mins.

TABLE 4

| | | | | Cell | | | | | |
|---|---|---|---|---|---|---|---|---|---|
| | Cell count | | | viability | Cell | Cell | ALP marker | | |
| | at P(1) | Cell count | Cell count | at P(1) | viability | viability | expression | ALP marker | ALP marker |
| | stage-UCB + | at P (1) | at P (1) | stage-UCB + | at P (1) | at P (1) | UCB + | expression | expression |
| | MB PL | stage-FBS | stage-PB-PL | MB PL | stage-FBS | stage-PB-PL | MB PL | FBS | PB-PL |
| Sample ID | (×10⁶) | (×10⁶) | (×10⁶) | (%) | (%) | (%) | (%) | (%) | (%) |
| Sample-01 | 14.28 | 14.34 | 13.98 | 97.95 | 98.52 | 96.54 | 90 | 94.9 | 89.2 |
| Sample-02 | 17.79 | 16.45 | 15.82 | 96.15 | 98.02 | 97.32 | 96 | 96.4 | 95.3 |
| Sample-03 | 17.73 | 13.08 | 12.95 | 95.97 | 96.55 | 94.99 | 92.2 | 87.3 | 86.9 |
| Sample-04 | 19.71 | 15.69 | 13.56 | 96.73 | 97.98 | 98.64 | 91 | 99.1 | 92.6 |
| Sample-05 | 16.23 | 12.28 | 11.47 | 96.09 | 95.89 | 95.37 | 90.5 | 93 | 90.2 |
| Sample-06 | 17.5 | 12.66 | 12.34 | 97.36 | 97.7 | 96.99 | 97.6 | 96.6 | 96.1 |
| Sample-07 | 17.76 | 19.18 | 18.49 | 98.59 | 98.3 | 97.59 | 90.1 | 93.1 | 91.1 |
| Sample-08 | 18.9 | 16.16 | 15.75 | 97.4 | 97.79 | 97.22 | 95.1 | 97.4 | 93.4 |
| Sample-09 | 18.83 | 8.6 | 8.9 | 96.73 | 96.56 | 96.15 | 94.4 | 96.8 | 92.4 |
| Sample-10 | 15.21 | 15.3 | 14.85 | 95.28 | 97.89 | 96.42 | 93.7 | 97.9 | 91.1 |
| Sample-11 | 17.36 | 21.06 | 18.32 | 96.2 | 98.61 | 98.58 | 92 | 93.9 | 92.2 |
| Sample-12 | 16.43 | 5.71 | 7.56 | 94.04 | 96.59 | 95.49 | 90.9 | 89.2 | 90.1 |
| Sample-13 | 15.6 | 15.78 | 14.67 | 93.87 | 98.06 | 92.21 | 92.8 | 81.1 | 89.2 |
| Sample-14 | 15.7 | 16.03 | 15.4 | 95.08 | 97.42 | 91.99 | 90.4 | 94.1 | 89.8 |
| Sample-15 | 14.39 | 17.68 | 15.66 | 96.65 | 98.52 | 94.39 | 93.4 | 91.6 | 90.8 |

TABLE 5

| S. No | Sample ID | Sterility | Mycoplasma | Endotoxin |
|---|---|---|---|---|
| 1 | Sample-01 | No growth | Negative | <0.750 EU/ml |
| 2 | Sample-02 | No growth | Negative | <0.750 EU/ml |
| 3 | Sample-03 | No growth | Negative | <0.750 EU/ml |
| 4 | Sample-04 | No growth | Negative | <0.750 EU/ml |
| 5 | Sample-05 | No growth | Negative | <0.750 EU/ml |
| 6 | Sample-06 | No growth | Negative | <0.750 EU/ml |
| 7 | Sample-07 | No growth | Negative | <0.750 EU/ml |
| 8 | Sample-08 | No growth | Negative | <0.750 EU/ml |
| 9 | Sample-09 | No growth | Negative | <0.750 EU/ml |
| 10 | Sample-10 | No growth | Negative | <0.750 EU/ml |
| 11 | Sample-11 | No growth | Negative | <0.750 EU/ml |
| 12 | Sample-12 | No growth | Negative | <0.750 EU/ml |
| 13 | Sample-13 | No growth | Negative | <0.750 EU/ml |
| 14 | Sample-14 | No growth | Negative | <0.750 EU/ml |
| 15 | Sample-15 | No growth | Negative | <0.750 EU/ml |

### Gene Expression Studies:

Total RNA was isolated from the cultured Mesenchymal stem cells and differentiated to osteoblasts cells by total RNA extraction method. Extracted RNA was quantified and using reverse transcriptase technique RNA was transcribed in the presence of oligo-dT primers for complementary DNA (cDNA) synthesis. The expression of different genes was assessed by using SYBR-Green RT-PCR master mix. Real-time PCR Master Mix containing, syber green probes, specific primers, and cDNA were mixed, and real-time RT-PCR was performed using a Rotar gene Q Real-Time RT-PCR. The primers used are shown in below table. Gene expressions was normalized to the reference gene GAPDH and calculated as the relative as the relative expression compared to control cells. Comparative analysis of the 12 different gene expression was done in cultured MSCs, differentiated pre-osteoblasts and mature osteoblasts with fold of gene expressions. Table 6 depicts the primer sequences for the genes which were analyzed.

TABLE 6

| Name of | | | |
|---|---|---|---|
| the Genes | Species | Forward (5′ → 3′) | Reverse (5′ → 3′) |
| OCT-4 | Human | GTTGATCCTCGGACCTGGCTA | GGTTGCCTCTCACTCGGTTCT |
| | | (SEQ ID NO: 1) | (SEQ ID NO: 2) |
| Nanog | Human | GTCTTCTGCTGAGATGCCTCACA | CTTCTGCGTCACACCATTGCTAT |
| | | (SEQ ID NO: 3) | (SEQ ID NO: 4) |
| Sox2 | Human | GCCGAGTGGAAACTTTTGTCG | GCAGCGTGTACTTATCCTTCTT |
| | | (SEQ ID NO: 5) | (SEQ ID NO: 6) |
| ALP | Human | ACC ATT CCC ACG TCT TCA CAT TT | AGA CAT TCT CTC GTT CAC CGC C |
| | | (SEQ ID NO: 7) | (SEQ ID NO: 8) |
| Collagen-1 | Human | GGACACAATGGATTGCAAGGCCGC | TAACCACTGCTCCACTCTGGATGG |
| | | (SEQ ID NO: 9) | (SEQ ID NO: 10) |
| RunX2 | Human | AGA TGA TGA CAC TGC CAC CTC TG | GGG ATG AAA TGC TTG GGA ACT |
| | | (SEQ ID NO: 11) | (SEQ ID NO: 12) |
| Osterix+ | Human | TAG TGG TTT GGG GTT TGT TTT ACC GC | AAC CAA CTC ACT CTT ATT CCC TAA GT |
| | | (SEQ ID NO: 13) | (SEQ ID NO: 14) |
| ICAM-1 | Human | GGCCGGCCAGCTTATACAC | TAGACACTTGAGCTCGGGCA |
| | | (SEQ ID NO: 15) | (SEQ ID NO: 16) |
| Leptin receptor | Human | AGGAAGCCCGAAGTTGTGTT | TCTGGTCCCGTCAATCTGA |
| | | (SEQ ID NO: 17) | (SEQ ID NO: 18) |
| Ephrin B2 | Human | GCATCTGTCTGCTTGGTCTTTATCAAC | ATGGCTGTGAGAAGGGACTCC |
| | | (SEQ ID NO: 19) | (SEQ ID NO: 20) |
| Ephrin B4 | Human | GAAGAAGGAGAGCTGTGTGGCAATC | GATGACTGTGAACTGTCCGTCGTT |
| | | (SEQ ID NO: 21) | (SEQ ID NO: 22) |
| MEPE | Human | CGAGTTTTCTGTGTGGGACTACTC | CTTAGTTTTCTCAGTCTGTGGTTGAAAT |
| | | (SEQ ID NO: 23) | (SEQ ID NO: 24) |

*Oligonucleotide sequences of sense (S) and antisense (A) primers used in the real-time PCR of target
and housekeeping gene.

Table 7 depicts List of RT-PCR markers used at P1 and P2 stages.

TABLE 7

| | | Pre− | | | Bone | |
|---|---|---|---|---|---|---|
| | | osteo- | Osteo- | Osteo- | lining | Osteo- |
| Markers | MSC | blast | blast | cyte | cells(BLC) | clast |
| OCT-4 | +++ | − | − | − | − | − |
| Nanog | +++ | − | − | − | − | − |
| Sox2 | +++ | − | − | − | − | − |
| ALP | − | + | +++ | − | | |
| Collagen- | − | ++ | +++ | − | | |
| 1 | | | | | | |
| RunX2 | | ++ | +++ | − | | |
| Ephrin | − | − | +++ | − | − | − |
| B4 | | | | | | |
| Osterix | − | ++ | +++ | − | − | − |
| MEPE | − | − | − | + | − | − |
| ICAM-1 | + | − | − | − | + | − |
| Leptin | + | − | − | − | + | − |
| receptor | | | | | | |
| Ephrin | − | − | − | −− | | + |
| B2 | | | | | | |

Table 8 depicts the difference in fold expression for different RT-PCR markers.

TABLE 8

| | Sample-01-Fold | | | Sample-02- Fold | | | Sample-03- Fold | | |
|---|---|---|---|---|---|---|---|---|---|
| | of expression | | | of expression | | | of expression | | |
| | | Pre- | Mature | | Pre- | Mature | | Pre- | Mature |
| | MSC | osteoblast | osteoblast | MSC | osteoblast | osteoblast | MSC | osteoblast | osteoblast |
| Genes | (P₀) | (P₁) | (P₂) | (P₀) | (P₁) | (P₂) | (P₀) | (P₁) | (P₂) |
| Oct-4 | 1 | 0.49 | 0.45 | 1 | 0.16 | 0.4 | 1 | 1.4 | 1.31 |
| Nanog | 1 | 1.15 | 0.98 | 1 | 1.18 | 0.74 | 1 | 1.18 | 1.31 |
| sox2 | 1 | 1.18 | 0.8 | 1 | 1.26 | 1.45 | 1 | 1.49 | 0.89 |
| ALP | 1 | 4 | 9 | 1 | 1 | 2 | 1 | 4.8 | 6.7 |
| Collagen- | 1 | 8 | 11 | 1 | 1 | 2 | 1 | 5.2 | 8.7 |
| 1 | | | | | | | | | |
| runx2 | 1 | 4 | 9.5 | 1 | 2 | 4 | 1 | 4.7 | 5.9 |
| OSTERIX | 1 | 8 | 13.5 | 1 | 2 | 4 | 1 | 3.7 | 4.5 |
| ICAM -I | 1 | 0 | 0 | 1 | 0 | 0 | 1 | 0.2 | 1 |
| leptin | 1 | 2 | 1.4 | 1 | 0.5 | 1 | 1 | 2.2 | 0.1 |
| ephrinB4 | 1 | 1.8 | 2.8 | 1 | 3.2 | 4.6 | 1 | 7.5 | 10.6 |
| MEPE | 1 | 2 | 2 | 1 | 2 | 2 | 1 | 6.8 | 5.9 |
| Ephrin-b2 | 1 | 0.4 | 0.3 | 1 | 0 | 0.3 | 1 | 0.3 | 0.8 |

### Overall Analysis of RT-PCR Expression Studies: Tables 7 and 8.

Oct4 Expression: At P0 stage shows higher expression compare to P1 (pre-osteoblast) and P2 (mature osteoblast) stage.

Nanog Expression: P0 stage shows expression and P1 stage little more expression than P0 but P2 stage shows lower expression than P0 stage.

SOX2 Expression: P0 stage shows expression and P1 stage little more expression than P0 but P2 stage shows lower expression than P0 stage.

ALP Expression: Compared to P0 stage, P1 and P2 stage shows higher expression Collagen-1 expression: Compared to P0 stage, P1 and P2 stage shows higher expression.

RunX2 expression: Compared to P0 stage, P1 and P2 stage shows higher expression Osterix Expression: Compared to P0 stage, P1 and P2 stage shows higher expression ICAM1 expression: P0 stage shows expression and very less expression can be observed at P1 & P2 stage.

Leptin expression: P0 shows some expression, and P1 stage little more expression than P0 but at P2 stage it showed lower than P0 stage.

Ephrin B4 expression: Compared to P0 stage, P1 and P2 stage showed higher expression.

MEPE Expression: Compared to P0 stage, P1 and P2 stage showed higher expression Ephrin b2 expression: At P0 stage shows certain expression and very less expression is observed at P1 and P2 stage.

The temporal expression of Oct-4, Nanog, SOX2, ALP, Collagen-1, RUNX2, OSTERIX, ICAM-I, Leptin, Ephrin B4, MEPE and Ephrin-B2 to evaluate osteoblastic phenotypic properties were studied. The gene expression results were similar with in vivo bone remodeling system. In general, the expression levels of collagen type I, RUNX2, OSTERIX and ALP were upregulated in the cell cultures in the differentiated and expansion medium of the invention. The expressions of alkaline phosphatase, RUNX2 and Osterix genes on the culture plates showed increasing levels with increasing culturing phase/time. The expression of Oct-4, Nanog, SOX2 seen in cultured MSCs were further downregulated in the differentiated osteoblasts cultures of the optimized medium. The expression of ICAM-I and Leptin were also downregulated. ICAM-I and Leptin markers of Bone lining cells are predominantly expressed in the fully formed bone cells in in vivo bone union. Thus, downregulation of these gene expression is a confirmatory result of obtaining adult, live, mature osteoblasts in the culture medium.

Therefore, from the present Example, it can be established that the osteoblast cells obtained from MSC as per the process as disclosed herein, leads to transplantation-ready osteoblast cells which are well differentiated. The gene expression results further establish that the osteoblast cells so obtained mimic the in-vivo bone remodeling system, therefore, providing ex-vivo cultured osteoblast cells having the potential to perform much like in-vivo cultured cells.

### Example 2

### Preparation of Osteoblast Cell-Mixture

A. Method of Preparation of PRP from Discarded Umbilical Cord Blood and Maternal Blood Plasma

### Part 1: Isolation of HSC's:

1. Umbilical Cord blood sample was collected at hospital and shipped to the processing facility of cord blood banking in controlled temperature at 18° C.-28° C.
2. The isolation of hematopoietic stem cells from cord blood was carried out within 72 hours from collection of cord blood.
3. Processing of umbilical cord blood sample was carried out under aseptic conditions in Biosafety cabinet.
4. Addition of sedimentation reagent into cord blood collection bag was done aseptically and then placed the collection bag on rocker for 5 minutes for proper mixing.
5. The cord blood collection bag was subjected for double sedimentation for 30-50 minutes under undisturbed conditions.
6. Leukocyte rich plasma was collected twice in processing bag after both sedimentations.
7. The cord blood bag containing leukocyte rich plasma was centrifuged at 1500-2100 rpm for 10-20 minutes.
8. After completion of centrifugation, the cord blood bag was placed on the Auto volume expresser in biosafety cabinet and the excess cord blood plasma was collected in a separate bag.
9. This excess cord blood plasma was used for preparation of fibrinogen and thrombin. Excess cord blood plasma was stored at 2-8° C. till processing.
10. Maternal whole blood sample of same cord blood sample was tested for Infectious diseases such as HIV, HCV, HBsAg and Syphilis Antibodies. Once confirmation of testing is received as negative for Infectious diseases then process the cord blood plasma for preparation of PRP.
11. After testing for Infectious diseases, maternal blood plasma was mixed with cord blood plasma and used for preparation of PRP.
   Part 2: Isolation of Platelets from UCB for the Preparation of PRP
   Part 3: Isolation of Platelets from MB for the Preparation of PRP

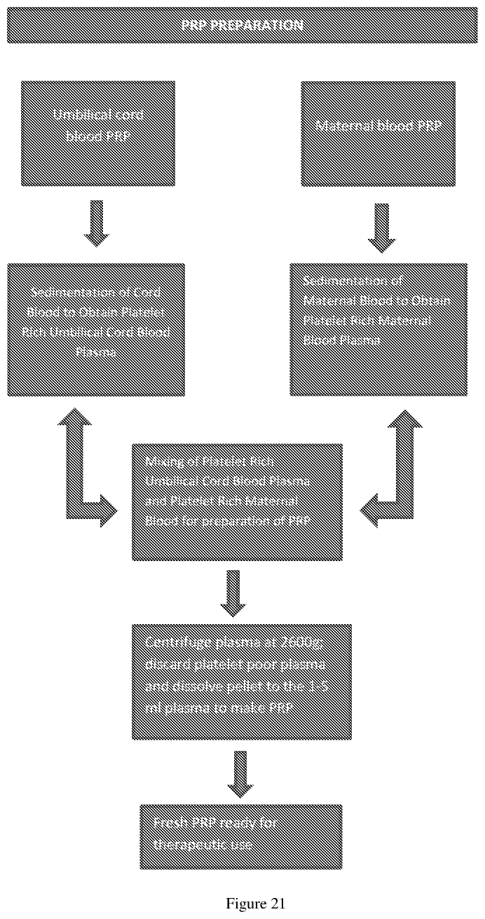

The combined process for preparing PRP from UCB and MB has been depicted in FIG. 21. The steps have been mentioned below:

To begin, at the cell processing centre, an aliquot of 100-200 μl were withdrawn for initial platelet count from the labelled umbilical cord blood (UCB) bag and maternal blood (MB) vacutainer tube.

For processing the umbilical cord blood (UCB) bag, a calculated volume of sedimentation reagent, such as Ficoll-Hipaque, Hespan®, Pentastarch, etc., was withdrawn under sterile conditions and injected into the umbilical cord blood (UCB) collection bag and kept on a rocking shaker for 5-10 minutes. A processing bag was labelled and attached to the said umbilical cord blood (UCB) collection bag. The said umbilical cord blood (UCB) collection bag was subjected to a first and then a second sedimentation for 35 minutes each so as to allow Rouleax formation of the red blood cells (RBCs) for better separation of the RBCs from the plasma. After said sedimentations, with the use of auto volume expresser, umbilical cord blood (UCB) platelet-rich plasma (PRP) was collected in the umbilical cord blood (UCB) processing bag. Further, the umbilical cord blood (UCB) platelet-rich plasma in the umbilical cord blood (UCB) processing bag was subjected to a third sedimentation for another 35 minutes to separate out hematopoietic stem cells (HSCs) from the plasma with the use of auto volume expresser and the final umbilical cord blood (UCB) derived platelet-rich plasma (PRP) was collected in a 50 ml conical tube.

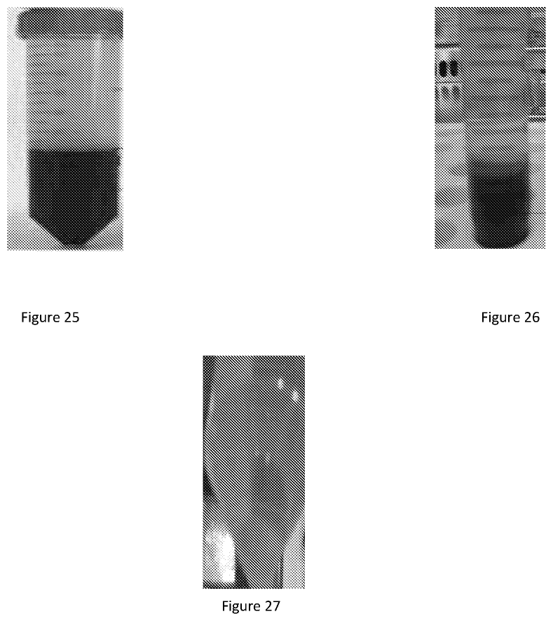

At the same time as the processing of umbilical cord blood (UCB), the maternal blood (MB) sample was kept for sedimentation for 60 minutes and then, the supernatant was collected to procure maternal blood (MB) derived platelet-rich plasma (PRP) into a 50 ml conical tube. FIGS. 25 and 26 represent the picture of sedimentation of umbilical cord blood to obtain platelet rich plasma (PRP) from umbilical cord blood and a picture of sedimentation of maternal blood to obtain platelet rich plasma (PRP) from maternal blood, respectively.

### Part 4: Mixing of Cord Blood and Maternal Blood PRP

The PRP from UCB and the PRP from MB were mixed as shown in FIG. 21. FIG. 27 depicts the picture of centrifuged PRP after mixing of Platelet Rich Umbilical Cord Blood Plasma and Platelet Rich Maternal Blood for preparation of PRP.

### Part 5: Concentration of UCB+MB PRP

The mixture as obtained in the previous step was centrifuged at 2600 g and the platelet poor plasma was discarded, and the respective pellet was dissolved in 1-5 ml of plasma to obtain the final PRP.

TABLE 9
Platelet recovery of prepared concentrated PRP
derived from discarded UCB and MB plasma

| | | Initial | Final | UCB + MB | | |
|---|---|---|---|---|---|---|
| Initial volume of | | Platelet | UCB + | platelet | Loss of | Platelet |
| discarded UCB + | | count | MB PRP | count | platelet | recovery |
| MB plasma | | (×10⁹/ml) | volume | (×10⁹/ml) | (%) | (%) |
| 10 ml | Sample 1 | 0.166 | 1.0 ml | 1.48 | 10.84 | 89.16 |
| | Sample 2 | 0.152 | | 1.38 | 09.21 | 90.79 |
| | Sample 3 | 0.173 | | 1.52 | 12.14 | 87.86 |
| | AVG | 0.164 | | 1.46 | 10.73 | 89.27 |
| 25 ml | Sample 1 | 0.165 | 2.5 ml | 1.38 | 16.36 | 83.64 |
| | Sample 2 | 0.138 | | 1.19 | 13.77 | 86.23 |
| | Sample 3 | 0.145 | | 1.24 | 14.48 | 85.52 |
| | AVG | 0.149 | | 1.27 | 14.87 | 85.13 |
| 50 ml | Sample 1 | 0.131 | 5.0 ml | 1.06 | 19.08 | 80.92 |
| | Sample 2 | 0.119 | | 0.98 | 17.65 | 82.35 |
| | Sample 3 | 0.152 | | 1.21 | 20.39 | 79.61 |
| | AVG | 0.134 | | 1.08 | 19.04 | 80.96 |

* Above plasma samples centrifuge at 2600 g for 10 min at 25° C. to obtain PRP

### B. Method for Preparing and Activating Live Cultured Osteoblast and Other Cell-Mixtures Using Freshly Prepared PRP Derived Umbilical Cord Blood and Maternal Blood Plasma for Therapeutic Use

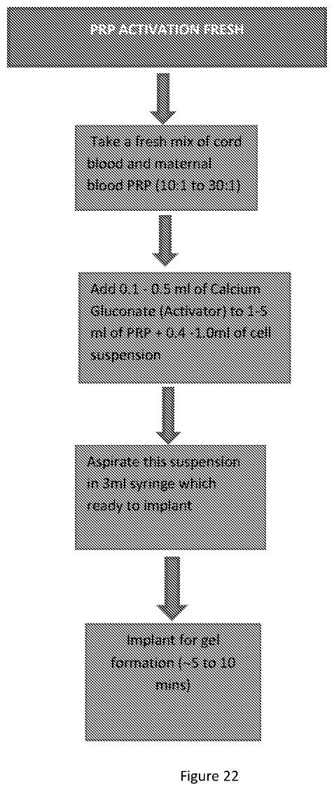

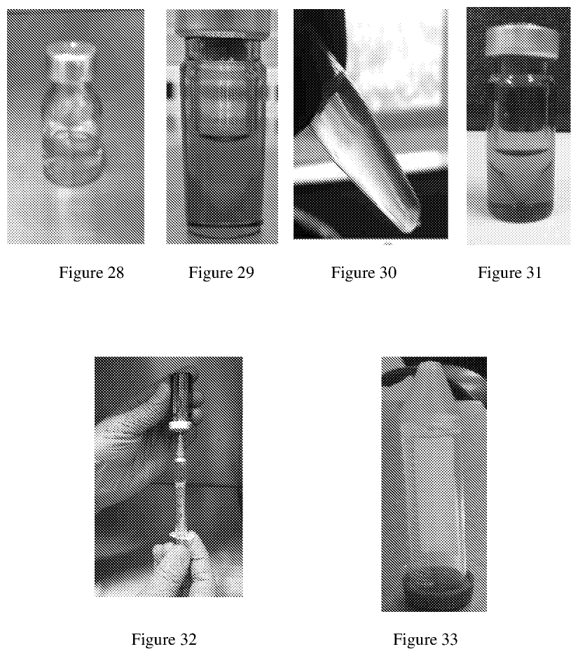

The method for preparing and activating the live cultured osteoblast cells is depicted in FIG. 28. The PRP of UCB and MB was mixed in a ratio range of 10:2 to 30:1, post which 0.1-0.5 ml calcium gluconate (activator) (FIG. 28) was added to the mixture along with 0.4-1.0 ml of osteoblast cell suspension (FIG. 29). FIG. 30 depicts the picture of PRP obtained by the process as described herein. FIGS. 31, 32, and 33 depict the stages of mixing, aspiration and the formation of gel, respectively using the PRP as disclosed herein. FIG. 22 shows a flowchart for preparing the gel using PRP.

### C. Method for Preparing and Activating Live Cultured Osteoblast and Other Cell-Mixtures Using Frozen and Thawed PRP (PL) Derived Umbilical Cord Blood and Maternal Blood Plasma for Therapeutic Use

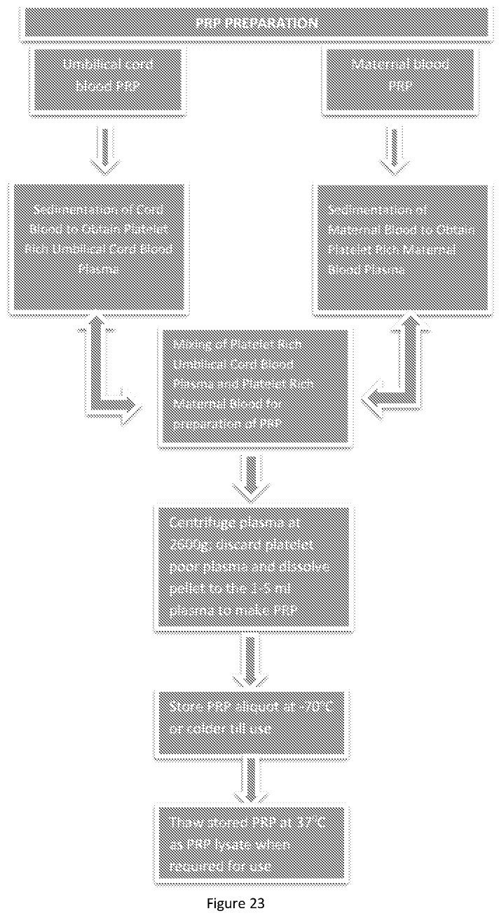

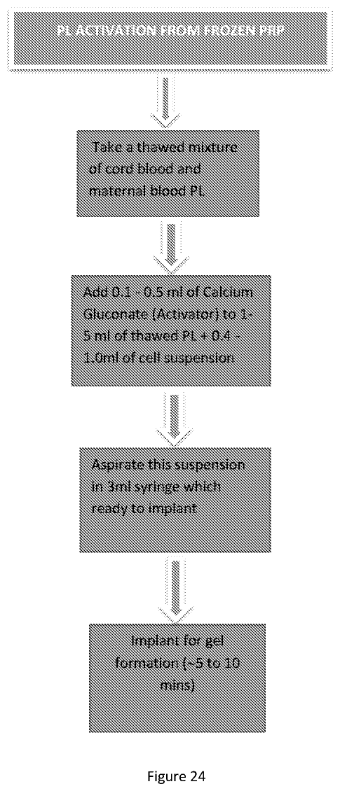

The method of obtaining frozen and thawed PRP (PL) and the method of activating live cultured cells by Frozen and thawed PRP (PL) has been depicted in FIG. 23 and FIG. 24, respectively.

### D. Comparison of Total Protein Content Between Freshly Prepared PRP and Frozen PRP (PL) of 6 and 12 Months.

TABLE 10
Comparison of protein content in the fresh PRP
with frozen PRP (PL) of 6 months and 12 months

| | Initial | Post 6-month | Post 12-month |
|---|---|---|---|
| | protein | protein | protein |
| | concentration | concentration | concentration |
| PRP | g/dL | [Frozen PRP (PL)] | [Frozen PRP (PL)] |
| sample | (Fresh PRP) | g/dL | g/dL |
| Sample 1 | 4.52 | 4.38 | 4.32 |
| Sample 2 | 4.85 | 4.78 | 4.68 |
| Sample 3 | 5.25 | 4.95 | 4.90 |

The data as shown in Table 10 shows that for all the three samples, the variation among the protein content was very minimal. Hence, even after freezing and thawing the protein content was intact.

E. Method of Preparation of Fibrinogen and Thrombin from Discarded Umbilical Cord Blood and Maternal Blood Plasma
Isolation of HSC's from Cord Blood Sample:
1. Umbilical Cord blood sample was collected at hospital and shipped to the processing facility of cord blood banking in controlled temperature at 18° C.-28° C.
2. The isolation of hematopoietic stem cells from cord blood was carried out within 72 hours from collection of cord blood.
3. Processing of umbilical cord blood sample was carried out under aseptic conditions in Biosafety cabinet.
4. Addition of sedimentation reagent into cord blood collection bag was done aseptically and then placed the collection bag on rocker for 5 minutes for proper mixing.
5. The cord blood collection bag was subjected for double sedimentation for 30-50 minutes under undisturbed conditions.
6. Leukocyte rich plasma was collected twice in processing bag after both sedimentations.
7. The cord blood bag containing leukocyte rich plasma was centrifuged at 1500-2100 rpm for 10-20 minutes.
8. After completion of centrifugation, the cord blood bag was placed on the Auto volume expresser in biosafety cabinet and the excess cord blood plasma was collected in a separate bag.
9. This excess cord blood plasma was used for preparation of fibrinogen and thrombin. Excess cord blood plasma was stored at 2-8° C. till processing.
10. Maternal whole blood sample of same cord blood sample was tested for Infectious diseases such as HIV, HCV, HBsAg and Syphilis Antibodies. Once confirmation of testing is received as negative for Infectious diseases then process the cord blood plasma for preparation of fibrinogen.
11. After testing for Infectious diseases, maternal blood plasma was mixed with cord blood plasma and used for preparation of fibrinogen.

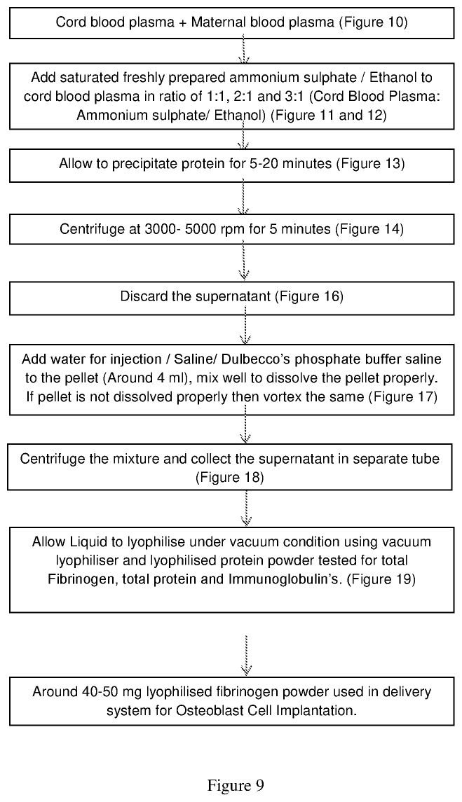

### Fibrinogen Isolation Procedure: (Depicted in FIG. 9)

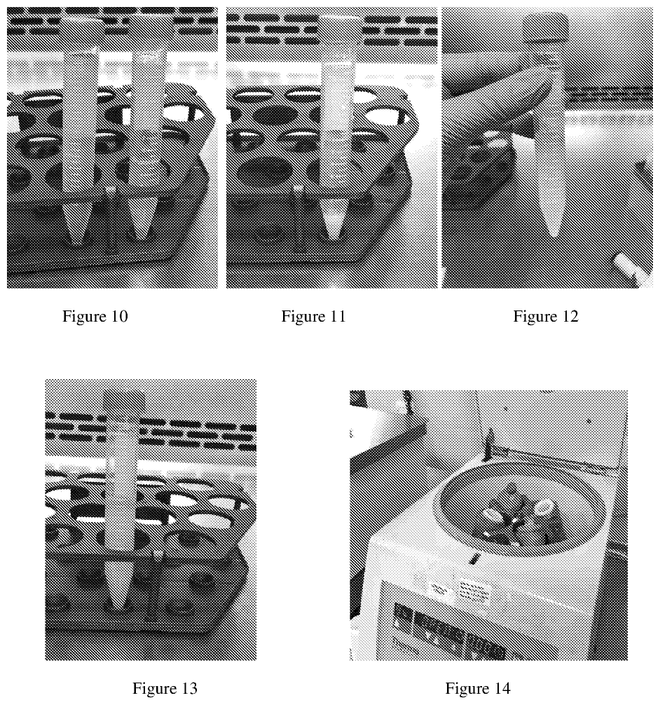

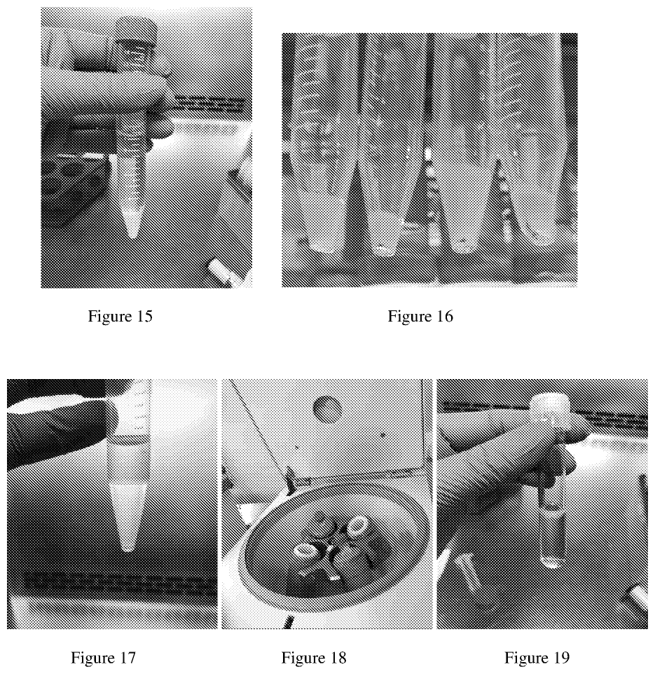

1. Additional cord blood plasma was mixed with maternal blood plasma after processing of cord blood sample is collected (FIG. 10).
2. Saturated Ammonium Sulphate/Ethanol was added to the cord blood plasma in ratio of 1:1, 2:1 or 3:1 (Cord blood plasma: Ammonium sulphate/Ethanol) (FIGS. 11 and 12) and allowed to precipitate the plasma proteins for 5-20 minutes (FIG. 13).
3. This mixture was centrifuged at 3000-5000 rpm for 5 minutes (FIG. 14) to settle down the protein precipitate in the form of pellet.
4. The supernatant was discarded (FIGS. 15 and 16) and around 4 ml of water for injection/Saline/Dulbecco's phosphate buffer saline was added to the pellet. Mixed well to dissolve the pellet properly (FIG. 17).
5. This mixture is centrifuged, and the supernatant was collected in separate tube (FIG. 18). This supernatant was used to prepare powdered form of fibrinogen.
6. Collected supernatant was allowed to lyophilize under vacuum conditions (FIG. 19) using vacuum lyophilizer and resultant lyophilized protein powder tested for total fibrinogen, total protein and Immunoglobulin's, if any.
7. 40-50 mg of lyophilized protein powder rich in fibrinogen was used in delivery system for osteoblast cell implantation.

### Thrombin Isolation Procedure:

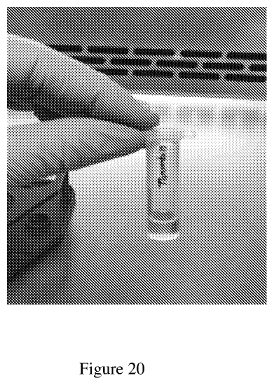

1. Additional Cord Blood plasma was mixed with maternal blood plasma after processing of cord blood sample is collected.
2. Saturated Ammonium Sulphate/Ethanol was added to the cord blood plasma in ratio of 1:1, 2:1 or 3:1 (Cord blood plasma: Ammonium sulphate/Ethanol) and allowed to precipitate the plasma proteins for 5-20 minutes.
3. This mixture was centrifuged at 3000-5000 rpm for 5 minutes and allowed to settle down the protein precipitate in the form of pellet.
4. The supernatant was discarded and around 4 ml of water for injection/Saline/Dulbecco's phosphate buffer saline was added to the pellet. Mixed well to dissolve the pellet properly.
5. This mixture was centrifuged, and the supernatant was collected in a separate tube. This supernatant was used to prepare powdered form of thrombin.
6. Collected supernatant was allowed to lyophilize under vacuum conditions using vacuum lyophiliser and lyophilized protein powder was tested for total thrombin and total protein.
7. Protein dissolved in Water for Injection (WFI)/saline/Dulbecco's phosphate buffer saline was used for the process of thrombin extraction.
8. Prothrombin was extracted by Ion exchange chromatography using Diethyl Aminoethyl (DEAE-IEC) followed by heparin Affinity Chromatography (A second DEAE-IEC step) followed by Immobilized Metal Affinity Chromatography (IMAC).
9. Collected Prothrombin was then activated to thrombin and purified by Hydrophobic Interaction Chromatography (HIC) and concentrated by Ultrafiltration.
10. This isolated thrombin (FIG. 20) is tested for total protein content and concentration of thrombin per microliter (μl).
11. 500 IU of isolated thrombin was mixed with cell and later on cord blood and maternal blood derived protein rich in fibrinogen and used for osteoblasts cell mixture delivery system.

### Method for Preparing Osteoblast Cell-Mixture Using Fibrinogen and Thrombin

For preparing the osteoblast cell-mixture, fibrinogen and thrombin used were obtained by the process as described herein.

The process for preparing the osteoblast cell-mixture comprises: (a) obtaining thrombin; (b) contacting thrombin with a first nutrient medium, to obtain a first mixture; (c) combining the first mixture with an osteoblast cell suspension, to obtain a second mixture; (d) obtaining fibrinogen; (e) contacting fibrinogen with a second nutrient medium, to obtain a third mixture; and (f) mixing the second mixture of step (c) with the third mixture of step (e), to obtain an osteoblast cell-mixture.

The specific process followed was as follows: (i) 1 ml of nutrient media was mixed with fibrin, and the contents were aspirated into syringe A; (ii) 1 ml of nutrient media was mixed with thrombin; (iii) 0.4 ml of osteoblast cell suspension (as described in Example 1) was mixed with 0.4 ml of thrombin (having nutrient media), and aspirated into syringe B; (iv) syringe A and B were fixed using Y-shaped dual syringe applicator and mixed, to obtain an osteoblast cell-mixture. The osteoblast cell-mixture comprises not less than 48 million osteoblast cells. In order to obtain a mixture as homogenous as possible and thus a homogenous final product, a stream of syringe A and B was simultaneously mixed.

One of the significant advantage of the process of preparing the cell mixture and transplanting the same in a subject is that it does not use additives like aprotinin and calcium chloride during the entire process.

The osteoblast cell-mixture thus obtained forms a gel in a time span of 3-6 minutes. The final volume of the cell suspension obtained by such mixture of fibrin glue along with the cultured adult live osteoblasts was 24 million in 1 ml of said composition. 1 ml of the fibrin glue amounted to two vials each of 12 million cells giving a total of 24 million cells. Further depending on the intensity of the defect site, more of the cells are supplemented. Thus, a total of 48 million cells are provided which constitutes to 12 million cells of each vial, 2 vials of fibrin powder reconstituted with the nutrient media, 2 vials of thrombin and 2 empty vials to prepare the said composition. Thus, the procedure involves a few thousand of the patients' mesenchymal stem cells' (MSC) are harvested from the bone marrow. Mesenchymal Stem cells are specialized cells present in the bone marrow of adults which have a capacity to replace a terminally differentiated cell derived from the mesenchyme with an identical cell type, herein the osteoblasts. The stem cells are harvested, cultured, multiplied and finally differentiated into adult live cultured osteoblast. These osteoblasts at the time of implantation have a number around 48 million cells. The dead bone is curetted out and is replaced with adult live cultured osteoblast cell suspension.

The present disclosure thus emphasizes a formulation wherein 4 ml of bone marrow aspirated from the iliac crest gives an average of 6.5-7.2×10⁶ nucleated cells. Differentiation of mesenchymal stem cells in vitro and giving rise to a range of osteoblast count of 50-70×10⁶ osteoblasts. The number of osteoblasts formed in 1 cm³ of bone is 4.07×10⁵ cells (Vashishth D, et al., “The Anatomical Record: An Official Publication of the American Association of Anatomists.” 2002 August; 267(4):292-5). The lowest number of osteoblasts that has been implanted is 12×10⁶ cells which gives rise to 30 cm³ (3.10 cm×3.10 cm×3.10 cm) of bone matrix. The highest number of osteoblasts that has been implanted is 48×10⁶ cells which gives rise to 117 cm³ (4.89 cm×4.89 cm×4.89 cm) of bone. Hence, by implantation of autologous adult live cultured osteoblasts the present disclosure has been demonstrated to have achieved new bone formation from 30 cm³ to 117 cm³. The osteoblast cell-mixture as disclosed herein has been shown to have been used for transplanting osteoblast cells in a subject in need. The preparation method as described in Example 2 can be applied not only for preparing osteoblast cell mixture but also to human autologous and allogenic stem cells, progenitor cells and including but not limited to chondrocytes, buccal epithelial cells, dermal cells, cardiomyocytes, neuronal, adipocyte, hepatocyte, islet cells.

### Example 3

### Treatment of Avascular Necrosis (AVN) Using the Osteoblast Cell-Mixture as Described Herein

AVN, regardless of the etiology, is characterized by diminished supply of progenitor osteoblasts, simultaneously pronounced activity of osteoclasts with resultant imbalance in bone remodeling. Osteoclasts play a role of scavenger in healthy bone; but in AVN, even immature osteocytes are resorbed due to enhanced activity of osteoclasts. This leads to greatly compromised bone quality, as bone generation is insufficient. The bone death and necrosis continue and disease condition progresses. Thus, cell-based treatment for AVN should aim to bring back the balance in remodeling by making available specifically osteoblasts that will overcome their short supply due to prevailing ischemic environment. These osteoblasts will regenerate fresh bone, like native bone.

As per the present Example, one of the treatment methodologies to treat AVN using the osteoblast cell-mixture (obtained by method as disclosed in Example 2) comprising osteoblast cells (obtained by method as disclosed in Example 1) comprises:
1. An incision was made on lateral side of the affected area.
2. Using a 7/9 mm sized reamer, single or multiple drill holes were made by standard core decompression method to approach the necrotic area
3. The necrotic bone was gently debrided with the help of long surgical scoops.
4. The table was tilted to bring the hip joint into gravity dependent position.
5. Long spinal needle was inserted in core decompression tunnel and tip was placed at defect area region under C-Arm guidance.
6. The osteoblast cell-mixture was injected at the defect site.
7. Waited for 8-10 minutes.
8. The site was closed with standard medical procedure of suturing.

### Case Study for Treatment of AVN

Primary Objective: To assess the safety of the Autologous Adult Live Cultured Osteoblasts implantation in avascular necrosis of hip joint(s).

Secondary Objective: To evaluate the efficacy of Autologous Adult Live Cultured Osteoblasts implantation in avascular necrosis of hip joint(s).

Number of patients: 14 patients were enrolled in the study. 14 patients completed the study.

Product detail: Osteoblast cell prepared as per the present disclosure (Autologous Adult Live Cultured Osteoblasts vial (0.4 mL))

Appearance: Colourless transparent vial product, which contains mixed precipitated pale-white-coloured autologous adult live cultured osteoblasts and red coloured fluid. This fluid becomes turbid when shaken.

Study Duration:

Total duration of participation for each subject in the study was approximately 28 weeks from the day of participation.

Total enrollment duration: Approximately 16 weeks

Total study duration: Approximately 44 weeks

### Criteria for Evaluation:

The safety endpoint: Incidence of adverse events (AEs) related to therapy

The efficacy endpoints are:

Change in Oxford Hip Score at visit 7 from baseline visit.

Change in Harris Hip Score at visit 7 from baseline visit.

Pain relief as per Visual Analogue Score (VAS) at visit 7 from baseline visit.

Change in MRI (Not less than 1.5 T MRI) at visit 7 from baseline visit

Change in X-ray at visit 7 from baseline visit.

Change in CT Scan at visit 7 from baseline visit.

### Efficacy Evaluation:

Change in Oxford Hip Score at visit 7 from baseline visit

TABLE 11

| | | Total | Total | | | |
|---|---|---|---|---|---|---|
| | | Oxford Hip | Oxford Hip | | Percentage | |
| Site | Patient | Score at | Score at | | Changes | Condition |
| ID | ID | Baseline | Visit 07 | Difference | (%) | (Effect) |
| O1 | 1 | 25 | 45 | 20 | 80.00 | Positive |
| O1 | 2 | 36 | 45 | 9 | 25.00 | Positive |
| O1 | 3 | 44 | 48 | 4 | 9.09 | Positive |
| O1 | 5 | 22 | 31 | 9 | 40.91 | Positive |
| O1 | 6 | 34 | 46 | 12 | 35.29 | Positive |
| O2 | 1 | 22 | 44 | 22 | 100.00 | Positive |
| O2 | 2 | 30 | 46 | 16 | 53.33 | Positive |
| O3 | 2 | 29 | 39 | 10 | 34.48 | Positive |
| O3 | 3 | 34 | 34 | 0 | 0.00 | Neutral |
| O3 | 5 | 22 | 48 | 26 | 118.18 | Positive |
| O4 | 1 | 36 | 41 | 5 | 13.89 | Positive |
| O4 | 2 | 37 | 43 | 6 | 16.22 | Positive |
| O4 | 4 | 29 | 43 | 14 | 48.28 | Positive |
| O4 | 5 | 28 | 35 | 7 | 25.00 | Positive |

| | | | | |
|---|---|---|---|---|
| Total Mean | 30.57 | 42.00 | 11.43 | 42.83 |
| Standard | 6.61 | 5.35 | 7.42 | |
| Deviation | | | | |

Conclusion: Overall change in Oxford Hip Score between baseline and visit 7 was noted as 42.83%, so it was concluded that there is highly statistically significant mean difference between baseline and visit 7 (Table 11).

Change in Harris Hip Score at visit 7 from baseline visit

TABLE 12

| | | Total | Total | | | |
|---|---|---|---|---|---|---|
| | | Harris Hip | Harris Hip | | Percentage | |
| Site | Patient | Score at | Score | Differ- | Changes | Condition |
| ID | ID | Baseline | Visit 07 | ence | (%) | (Effect) |
| O1 | 1 | 52 | 93 | 41 | 78.85 | Positive |
| O1 | 2 | 66 | 95 | 29 | 43.94 | Positive |
| O1 | 3 | 82 | 100 | 18 | 21.95 | Positive |
| O1 | 5 | 63 | 89 | 26 | 41.27 | Positive |
| O1 | 6 | 60 | 94 | 34 | 56.67 | Positive |
| O2 | 1 | 83 | 96 | 13 | 15.66 | Positive |
| O2 | 2 | 77 | 86 | 9 | 11.69 | Positive |
| O3 | 2 | 51 | 74 | 23 | 45.10 | Positive |
| O3 | 3 | 64 | 64 | 0 | 0.00 | Neutral |
| O3 | 5 | 58 | 100 | 42 | 72.41 | Positive |
| O4 | 1 | 89 | 93 | 4 | 4.49 | Positive |
| O4 | 2 | 75 | 93 | 18 | 24.00 | Positive |
| O4 | 4 | 62 | 89 | 27 | 43.55 | Positive |
| O4 | 5 | 55 | 87 | 32 | 58.18 | Positive |

| | | | | |
|---|---|---|---|---|
| Total Mean | 66.93 | 89.50 | 22.57 | 36.98 |
| Standard | 12.20 | 9.84 | 12.95 | |
| Deviation | | | | |

Conclusion: Overall change in Harris Hip Score between baseline and visit 7 was noted as 36.98%, so it was concluded that there is highly statistically significant mean difference between baseline and visit 7 (Table 12).

Pain Relief as Per Visual Analogue Score (VAS) at Visit 7 from Baseline Visit.

TABLE 13

| | | Total | Total | | | |
|---|---|---|---|---|---|---|
| | | Visual | Visual | | | |
| | | Analogue | Analogue | | Percentage | |
| Site | Patient | Score at | Score at | Differ- | Changes | Condition |
| ID | ID | Baseline | Visit 07 | ence | (%) | (Effect) |
| O1 | 1 | 40 | 10 | −30 | −75.00 | Positive |
| O1 | 2 | 15 | 10 | −5 | −33.33 | Positive |
| O1 | 3 | 20 | 0 | −20 | −100.00 | Positive |
| O1 | 5 | 30 | 80 | 50 | 166.67 | Negative |
| O1 | 6 | 40 | 30 | −10 | −25.00 | Positive |
| O2 | 1 | 80 | 30 | −50 | −62.50 | Positive |
| O2 | 2 | 80 | 20 | −60 | −75.00 | Positive |
| O3 | 2 | 30 | 20 | −10 | −33.33 | Positive |
| O3 | 3 | 40 | 40 | 0 | 0.00 | Neutral |
| O3 | 5 | 40 | 0 | −40 | −100.00 | Positive |
| O4 | 1 | 40 | 5 | −35 | −87.50 | Positive |
| O4 | 2 | 50 | 10 | −40 | −80.00 | Positive |
| O4 | 4 | 60 | 10 | −50 | −83.33 | Positive |
| O4 | 5 | 70 | 15 | −55 | −78.57 | Positive |

| | | | | |
|---|---|---|---|---|
| Total Mean | 45.36 | 20.00 | −25.36 | −47.64 |
| Standard | 20.42 | 20.85 | 29.19 | |
| Deviation | | | | |

Conclusion: Overall change in VAS Score between baseline and visit 7 was noted as −47.64%, so it was concluded that there is highly statistically significant mean difference between baseline and visit 7 (Table 13).

In CT-scan/MRI/X-ray studies at visit 7 (24 weeks post implant) compared to baseline recording suggest, there was marginal decrease in necrotic tissue at the affected area of AVN in seven (07) of the 14 randomized patients. Remaining patients had preservation of architecture of affected part without any worsening of the joint treated with osteoblast cells of the present disclosure. The possible reason for only marginal improvement is due to the complex nature and long process involved in osteogenesis.

Safety Evaluation: During the study period, out of 14 patients, total 04 adverse events were reported in three (03) different patients. Among the reported AEs, two events (i.e., fever and swelling on foot) were of grade 02 (moderate) and one AE (asymptomatic urinary tract infection) was of mild severity. All these AEs which were not related and were resolved on symptomatic treatment to the implantation.

One patient reported serious adverse event of grade 03 (Severe). Patient with SAE had sub trochanteric fracture of right proximal femur because of accidental fall which was not considered to be related with the implantation. This patient was operated in next follow up and necessary post-operative care was provided to the patient.

Conclusion: Based on the safety and efficacy results derived at 24 weeks post osteoblasts implantation in 14 patients, it is concluded that osteoblasts is safe and effective treatment option for Avascular Necrosis (AVN) patients in improving quality of life and management of pain.

### Example 4

### Treatment of Non-Union Fractures Using the Osteoblast Cell-Mixture

A non-union is a fracture in bone that has not healed even at 9 months post routine treatment. The loose ends of the non-union will have compromised blood supply and it is documented that the bone resorption in this area is about 50 times higher as compared to that in healthy bone. This clearly means that osteoblast activity required for bone regeneration is greatly compromised. Thus, in many non-union, the initial gap at the fracture site widens. If locally osteoblasts are supplied, they can reverse the imbalance in bone remodeling, support new bone formation and heal the fracture.

The surgical method of use of osteoblast cell-mixture as disclosed in the present disclosure for treatment of a non-union in any bone will vary depending on the bone and the success of any previous surgery. As per the present Example, one of the treatment methodologies to treat non-union fractures is using the osteoblast cell-mixture (obtained by method as disclosed in Example 2) comprising osteoblast cells (obtained by method as disclosed in Example 1).

Osteoblast cells can be implanted during a corrective surgery in an old non-union, where previous surgery has failed (e.g., wrong plating or nailing or screwing) or any additional plating etc. is required to be done, the non-union site is cut open using a scalpel/knife. The musculature is set apart while bleeding is kept under control. The correction procedure is performed. The gap in the exposed non-union is implanted with semi-solid osteoblasts suspension following osteoblasts preparation method. The incision is serially sutured and closed inside-out. During a process of corrective cosmetic/plastic surgery many times, a non-union that is operated upon frequently, requires cosmetic correction. During such skin grafting procedures, the incision made can be used to access the non-union region of the concerned bone. The gap in the exposed non-union is implanted with semi-solid osteoblasts suspension following its preparation method. The topical cosmetic surgery is done alongside the implantation. As an isolated minimally invasive procedure in absence of any other complication or abnormality, and when there is no other revision or corrective surgery, the non-union can be located using an X-ray as a reference. Osteoblast cell-mixture prepared following standard protocol, can be injected through intradermal route.

### Example 5

### Treatment of Oral and Maxillofacial Abnormalities Using Osteoblast Cell-Mixture as Disclosed in the Present Disclosure

Bone is a dynamic tissue, and if it not put to use it starts regressing. Edentulous bone loss is an example, and about 50% of dental implantation processes require bone augmentation as a prerequisite. Like any other condition, in OMF conditions also, the bone remodeling is compromised with bone resorption pronounced as compared to simultaneous bone regeneration. Here, at cellular level, recruitment of osteoblasts and their further differentiation into osteocytes to form firm bone is imbalanced, resulting in relatively enhanced osteoclast activity that results in bone loss. Thus, supplementing the affected OMF region with cultured osteoblasts would be expected to give promising results.

Many conditions with loss of teeth end up with bone loss to the extent that dental implant placing is not possible without attaining enough height, length and width of alveolar bone. As per the present Example, one of the treatment methodologies to treat oral and maxillofacial abnormalities is using the osteoblast cell-mixture (obtained by method as disclosed in Example 2) comprising osteoblast cells (obtained by method as disclosed in Example 1).

Mandibular bone augmentation—The affected length of the mandible was accessed by placing cheek protractor. The supportive use of bone graft and/or membrane and/or scaffold and/or titanium mesh is dependent on the height and width of mandible to be achieved. Surgical scalpel was used to make the first incision and to cut the flap. This will split open the mandibular ridge to make space for implant. By using appropriate drills, implant guide was placed.

The slit edges were used for placing the scaffold and membrane or titanium mesh and bone graft. Titanium mesh was fixed with screws, depending on the area covered. Underneath the mesh, the bone graft material soaked in semi-solid osteoblasts suspension (cell mixture) was fill-packed. The rest of the osteoblasts suspension was instilled over the bone graft material and ensured that it sets perfect.

Primary suturing was followed by secondary suturing to close the incision.

Once it was ensured that enough height and width of bone is achieved, the mesh was removed, and dental implants can be placed as appropriate.

### Example 6

### Treatment of Bone Cyst Using the Osteoblast Cell-Mixture as Disclosed in the Present Disclosure

Bone cysts are common in young adolescents, and most often are idiopathic, although, the possible malignant expression cannot be negated. Surgical excision is mandatory as is the replacement of lost bone. Other conventional methods like bone grafting, either vascular or avascular, doesn't lead to complete integration of the grafted bone with the native bone. It also leaves gaps as hard grafted bone doesn't occupy the space available, and this leads to compromised strength and movement of the bone. As the bone is lost, replacing it with osteogenic cell population allowing bone regeneration with native bone morphology and other qualities will help regain bone function completely. As per the present Example, one of the treatment methodologies to treat bone cyst is using the osteoblast cell-mixture (obtained by method as disclosed in Example 2) comprising osteoblast cells (obtained by method as disclosed in Example 1).

### Advantages of the Present Disclosure:

The osteoblast cell-mixture as disclosed in the present disclosure shall be of significant advantage in terms of the encouraging clinical study that has been described in the present disclosure. The osteoblast cell-mixture ensures uniform coverage over the damaged site and has a gelling time of 3-6 minutes that ensures minimal unwanted spread of the osteoblast cell-mixture. Further, the method as disclosed in the present disclosure for preparing the osteoblast cell-mixture is fairly simple and avoids the usage of additives like aprotinin and calcium chloride which are generally used in preparing such cell mixtures. By avoiding the usage of additives, the process as disclosed in the present disclosure provides economic significance over the traditional processes and also minimizes the usage of additives in the composition that needs to be transplanted in a subject. The state-of-the-art technique exploits usage of certain additives to achieve the desired result, but the present disclosure provides a solution by avoiding the additives. Also, the timeline of recovery of a subject undergoing treatment using a process as disclosed herein comprising the osteoblast cell-mixture as disclosed herein has been found to be encouraging. The present disclosure also discloses a method for obtaining the osteoblast cell-mixture which are ready for transplantation, wherein the method involves direct steps of mixing the osteoblast cells with platelet lysate or PRP as disclosed herein, thereby even avoiding separate addition of thrombin and fibrinogen to the process steps. Therefore, following the methods as disclosed herein can be effective in terms of ease of usage and in terms of cost effectiveness.

By exploiting the process of transplantation as disclosed herein, complete bone regeneration is achieved in a subject, also, it provides a platform for transplanting autogenic osteoblast cells.

I/We claim:

1. A method for preparing an osteoblast cell-mixture, said method comprising:
a) obtaining thrombin;
b) contacting thrombin with a first nutrient medium, to obtain a first mixture;
c) combining the first mixture with an osteoblast cell suspension, to obtain a second mixture;
d) obtaining fibrinogen;
e) contacting fibrinogen with a second nutrient medium, to obtain a third mixture; and
f) mixing the second mixture of step (c) with the third mixture of step (e), to obtain an osteoblast cell-mixture.

2. A method of delivering osteoblast cells into a subject, said method comprising:
a) obtaining thrombin;
b) contacting thrombin with a first nutrient medium, to obtain a first mixture;
c) combining the first mixture with an osteoblast cell suspension, to obtain a second mixture;
d) obtaining fibrinogen;
e) contacting fibrinogen with a second nutrient medium, to obtain a third mixture;
f) mixing the second mixture of step (c) with the third mixture of step (e), to obtain an osteoblast cell-mixture; and
g) delivering the osteoblast cell-mixture to a site in a subject.

3. A method of delivering osteoblast cells into a subject, said method comprising:
a) obtaining thrombin;
b) contacting thrombin with a first nutrient medium, to obtain a first mixture;
c) combining the first mixture with an osteoblast cell suspension, to obtain a second mixture;
d) obtaining fibrinogen;
e) contacting fibrinogen with a second nutrient medium, to obtain a third mixture;
f) mixing the second mixture of step (c) with the third mixture of step (e), to obtain an osteoblast cell-mixture;
g) incising lateral side of an affected area in a subject to create an incision;
h) drilling holes through the incision to gain access to a defect area;
i) adjusting position of the subject in gravity-dependent position;
j) injecting the osteoblast cell-mixture of step (f) into the defect area and allowing the cell mixture to form a gel network; and
k) applying standard medical procedures for suturing, to deliver osteoblast cell-mixture into the subject.

4. A method for preparing an osteoblast cell-mixture, said method comprising:
a) obtaining umbilical cord blood (UCB) derived platelet rich plasma;
b) obtaining maternal blood (MB) derived platelet rich plasma;
c) contacting umbilical cord blood (UCB) derived platelet rich plasma and maternal blood (MB) derived platelet rich plasma in a ratio range of 10:1 to 30:1, to obtain a platelet rich plasma (PRP) mix;
d) obtaining at least one activator; and
e) contacting the PRP mix, and the at least one activator with an osteoblast cell suspension, to obtain the osteoblast cell-mixture.

5. A method of delivering osteoblast cells into a subject, said method comprising:
a) obtaining umbilical cord blood (UCB) derived platelet rich plasma;
b) obtaining maternal blood (MB) derived platelet rich plasma;
c) contacting umbilical cord blood (UCB) derived platelet rich plasma and maternal blood (MB) derived platelet rich plasma in a ratio range of 10:1 to 30:1, to obtain a platelet rich plasma (PRP) mix;
d) obtaining at least one activator;
e) contacting the PRP mix, and the at least one activator with an osteoblast cell suspension, to obtain an osteoblast cell-mixture; and
f) delivering the osteoblast cell-mixture to a site in a subject.

6. A method of delivering osteoblast cells into a subject, said method comprising:
a) obtaining umbilical cord blood (UCB) derived platelet rich plasma;
b) obtaining maternal blood (MB) derived platelet rich plasma;
c) contacting umbilical cord blood (UCB) derived platelet rich plasma and maternal blood (MB) derived platelet rich plasma in a ratio range of 10:1 to 30:1, to obtain a platelet rich plasma (PRP) mix;
d) obtaining at least one activator;
e) contacting the PRP mix, and the at least one activator with an osteoblast cell suspension, to obtain an osteoblast cell-mixture;
f) incising lateral side of an affected area in a subject to create an incision;
g) drilling holes through the incision to gain access to a defect area;
h) adjusting position of the subject in gravity-dependent position;
i) injecting the osteoblast cell-mixture of step (e) into the defect area and allowing the cell mixture to form a gel network; and
j) applying standard medical procedures for suturing, to deliver osteoblast cell-mixture into the subject.

7. A method for preparing an osteoblast cell-mixture, said method comprising:
a) obtaining a platelet lysate comprising a lysate obtained from a mixture of an umbilical cord blood (UCB) derived platelet rich plasma and a maternal blood (MB) derived platelet rich plasma;
b) obtaining at least one activator; and
c) contacting the platelet lysate and the at least one activator with an osteoblast cell suspension, to obtain the osteoblast cell-mixture.

8. A method of delivering osteoblast cells into a subject, said method comprising:
a) obtaining a platelet lysate comprising a lysate obtained from a mixture of an umbilical cord blood (UCB) derived platelet rich plasma and a maternal blood (MB) derived platelet rich plasma;
b) obtaining at least one activator;
c) contacting the platelet lysate and the at least one activator with an osteoblast cell suspension, to obtain the osteoblast cell-mixture; and
d) delivering the osteoblast cell-mixture to a site in a subject.

9. A method of delivering osteoblast cells into a subject, said method comprising:
a) obtaining a platelet lysate comprising a lysate obtained from a mixture of an umbilical cord blood (UCB) derived platelet rich plasma and a maternal blood (MB) derived platelet rich plasma;
b) obtaining at least one activator;
c) contacting the platelet lysate and the at least one activator with an osteoblast cell suspension, to obtain the osteoblast cell-mixture;
d) incising lateral side of an affected area in a subject to create an incision;
e) drilling holes through the incision to gain access to a defect area;
f) adjusting position of the subject in gravity-dependent position;
g) injecting the osteoblast cell-mixture of step (c) into the defect area and allowing the cell mixture to form a gel network; and
h) applying standard medical procedures for suturing, to deliver osteoblast cell-mixture into the subject.

10. The method as claimed in any one of the claims 1 to 9, wherein the osteoblast cell suspension is prepared from a mesenchymal stem cell suspension, and said mesenchymal stem cell suspension is obtained from a bone marrow sample.

11. The method as claimed in claim 10, wherein the mesenchymal stem cell suspension is cultured in presence of a nutrient medium comprising a platelet lysate to obtain the osteoblast cell suspension.

12. The method as claimed in claim 11, wherein the platelet lysate comprises a lysate obtained from a mixture of an umbilical cord blood (UCB) derived platelet rich plasma and a maternal blood (MB) derived platelet rich plasma.

13. The method as claimed in claim 12, wherein the mixture of an umbilical cord blood (UCB) derived platelet rich plasma and a maternal blood (MB) derived platelet rich plasma comprises 0.3×10⁹ to 1.5×10⁹ platelets/ml.

14. The method as claimed in any one of the claims 1 to 3, wherein the first mixture comprises 100-600 IU/ml thrombin.

15. The method as claimed in any one of the claims 1 to 3, wherein the third mixture comprises 20-100 mg/ml of fibrinogen.

16. The method as claimed in any one of the claims 1 to 3, wherein the first nutrient medium of step (b) or the second nutrient medium of step (e) comprises at least one medium selected from a group consisting of DMEM, αMEM, IMDM, and combinations thereof.

17. The method as claimed in any one of the claims 1 to 9, wherein the osteoblast cell-mixture is used in transplantation of osteoblast cells into a subject.

18. The method as claimed in any one of the claims 1 to 9, wherein the osteoblast cell suspension comprises osteoblast cells in a range of 12×10⁶ cells to 60×10⁶ cells.

19. The method as claimed in claim 2, wherein the mixing of the second mixture and the third mixture, and the delivering of the osteoblast cell-mixture is done by a dual syringe device.

20. The method as claimed in claim 3, wherein the injecting of the osteoblast cell-mixture is done by a dual syringe device.

21. The method as claimed in any one of the claims 1 to 3, wherein the second mixture and the third mixture is mixed in a ratio having a range of 1:0.5 to 1:2.

22. The method as claimed in any one of the claim 2, 3, 5, 6, 8, or 9 wherein the method achieves bone regeneration.

23. The method as claimed in any one of the claims 1 to 3, wherein fibrinogen is obtained from a mixture of umbilical cord blood (UCB) plasma and maternal blood (MB) plasma.

24. The method as claimed in any one of the claims 1 to 3, wherein thrombin is obtained from a mixture of umbilical cord blood (UCB) plasma and maternal blood (MB) plasma.

25. The method as claimed in any one of the claim 1, 2, 4, 5, 7, or 8, wherein the osteoblast cell-mixture forms a gel network in a time in a range of 2 seconds-6 minutes.

26. The method as claimed in any one of the claim 3, 6, or 9, wherein the gel network is formed in a time in a range of 2 seconds-6 minutes.

27. The method as claimed in any one of the claim 3, 6, or 9, wherein in the defect area, curettage is done to remove necrosed bone, and a saline wash is given through the holes.

28. The method as claimed in any one of the claims 1 to 9, wherein the osteoblast cell-mixture comprises autologous osteoblast cells.

29. The method as claimed in any one of the claim 2, 3, 5, 6, 8, or 9, wherein the method is used to treat conditions selected from a group consisting of non-union fracture, fibrous dysplasia, avascular necrosis, oral and maxillofacial fractures, and sinus lift.

30. The method as claimed in any one of the claims 4 to 9, wherein the at least one activator is selected from a group consisting of calcium gluconate, calcium saccharate, deoxy gluconate, calcium glucoheptonate, calcium chloride, and combinations thereof.


SEQUENCE LISTING (24 sequences)

SEQ ID NO: 1 - DNA, 21 bp, Artificial Sequence
  Forward Primer sequence for OCT-4
gttgatcctcggacctggcta

SEQ ID NO: 2 - DNA, 21 bp, Artificial Sequence
  Reverse Primer sequence for OCT-4
ggttgcctctcactcggttct

SEQ ID NO: 3 - DNA, 23 bp, Artificial Sequence
  Forward Primer sequence for Nanog
gtcttctgctgagatgcctcaca

SEQ ID NO: 4 - DNA, 23 bp, Artificial Sequence
  Reverse Primer sequence for Nanog
cttctgcgtcacaccattgctat

SEQ ID NO: 5 - DNA, 21 bp, Artificial Sequence
  Forward Primer sequence for Sox2
gccgagtggaaacttttgtcg

SEQ ID NO: 6 - DNA, 22 bp, Artificial Sequence
  Reverse Primer sequence for Sox2
gcagcgtgtacttatccttctt

SEQ ID NO: 7 - DNA, 23 bp, Artificial Sequence
  Forward Primer sequence for ALP
accattcccacgtcttcacattt

SEQ ID NO: 8 - DNA, 22 bp, Artificial Sequence
  Reverse Primer sequence for ALP
agacattctctcgttcaccgcc

SEQ ID NO: 9 - DNA, 24 bp, Artificial Sequence
  Forward Primer sequence for Collagen-1
ggacacaatggattgcaaggccgc

SEQ ID NO: 10 - DNA, 24 bp, Artificial Sequence
  Reverse Primer sequence for Collagen-1
taaccactgctccactctggatgg

SEQ ID NO: 11 - DNA, 23 bp, Artificial Sequence
  Forward Primer sequence for RunX2
agatgatgacactgccacctctg

SEQ ID NO: 12 - DNA, 21 bp, Artificial Sequence
  Reverse Primer sequence for RunX2
gggatgaaatgcttgggaact

SEQ ID NO: 13 - DNA, 26 bp, Artificial Sequence
  Forward Primer sequence for Osterix
tagtggtttggggtttgttttaccgc

SEQ ID NO: 14 - DNA, 26 bp, Artificial Sequence
  Reverse Primer sequence for Osterix
aaccaactcactcttattccctaagt

SEQ ID NO: 15 - DNA, 19 bp, Artificial Sequence
  Forward Primer sequence for ICAM-1
ggccggccagcttatacac

SEQ ID NO: 16 - DNA, 20 bp, Artificial Sequence
  Reverse Primer sequence for ICAM-1
tagacacttgagctcgggca

SEQ ID NO: 17 - DNA, 20 bp, Artificial Sequence
  Forward Primer sequence for Leptin receptor
aggaagcccgaagttgtgtt

SEQ ID NO: 18 - DNA, 19 bp, Artificial Sequence
  Reverse Primer sequence for Leptin receptor
tctggtcccgtcaatctga

SEQ ID NO: 19 - DNA, 27 bp, Artificial Sequence
  Forward Primer sequence for Ephrin B2
gcatctgtctgcttggtctttatcaac

SEQ ID NO: 20 - DNA, 21 bp, Artificial Sequence
  Reverse Primer sequence for Ephrin B2
atggctgtgagaagggactcc

SEQ ID NO: 21 - DNA, 25 bp, Artificial Sequence
  Forward Primer sequence for Ephrin B4
gaagaaggagagctgtgtggcaatc

SEQ ID NO: 22 - DNA, 24 bp, Artificial Sequence
  Reverse Primer sequence for Ephrin B4
gatgactgtgaactgtccgtcgtt

SEQ ID NO: 23 - DNA, 24 bp, Artificial Sequence
  Forward Primer sequence for MEPE
cgagttttctgtgtgggactactc

SEQ ID NO: 24 - DNA, 28 bp, Artificial Sequence
  Reverse Primer sequence for MEPE
cttagttttctcagtctgtggttgaaat